(19) United States
(12) Patent Application Publication
(10) Pub. No.: US 2020/0261640 A1
(43) Pub. Date: Aug. 20, 2020

(54) METHOD FOR TREATING AN ARTERIOVENOUS FISTULA (AVF) DILATED VENOUS SEGMENT IN A HEMODIALYSIS PATIENT WHOSE ARTERIOVENOUS FISTULA (AVF) EXHIBITS ABNORMALLY HIGH VENOUS BLOOD FLOW OR/AND PRESSURE

(71) Applicant: VASCULAR GRAFT SOLUTIONS LTD, Tel-Aviv (IL)

(72) Inventor: Eyal ORION, Ramat Efal (IL)

(21) Appl. No.: 16/794,580

(22) Filed: Feb. 19, 2020

Related U.S. Application Data
(60) Provisional application No. 62/807,931, filed on Feb. 20, 2019.

(51) Int. Cl. A61M 1/36; A61F 2/82; A61B 17/11; A61B 5/026; A61B 5/02

(52) U.S. Cl. CPC A61M 1/3655; A61F 2/82; A61B 17/11; A61B 2017/1132; A61B 5/02007; A61B 2017/1107; A61B 5/026

(57) ABSTRACT

Method for treating an arteriovenous fistula dilated venous segment in a hemodialysis patient whose arteriovenous fistula exhibits abnormally high venous blood flow or/and pressure. Includes: surgically reconstructing the AVF dilated venous segment, to form an AVF reconstructed venous segment, followed by covering and supporting the AVF reconstructed venous segment with implantable blood vessel external support, such that the arteriovenous fistula will exhibit normal venous blood flow and pressure. Also disclosed is a method for pre-determining an arteriovenous fistula reconstructed venous segment lumen diameter, as guidance for treating AVF dilated venous segments in hemodialysis patients. Also disclosed is use of an implantable blood vessel external support for treating a hemodialysis AVF dilated venous segment. Applicable for preventing or treating various pathological conditions (progressive heart disease, venous aneurysms, venous intimal hyperplasia, venous thrombosis, and bulges of the arm) in hemodialysis patients that are commonly associated with AVF dilated venous segments.

### CROSS-REFERENCE TO RELATED APPLICATIONS

This application claims the benefit of U.S. Provisional Patent Application Ser. No. 62/807,931, filed Feb. 20, 2019, the entire contents of which are herein incorporated by reference.

### FIELD OF THE INVENTION

The present invention, in some embodiments thereof, relates to the field of hemodialysis, particularly regarding treating abnormal vasculature and hemodynamic type phenomena associated with hemodialysis. More particularly, but not exclusively, the present invention relates to a method for treating an arteriovenous fistula dilated venous segment in a hemodialysis patient whose arteriovenous fistula exhibits abnormally high venous blood flow or/and pressure. The present invention is applicable for preventing or treating various pathological conditions (such as progressive heart disease, venous aneurysms, venous intimal hyperplasia, venous thrombosis, and unseemly appearance (i.e., bulges) of the arm) in a hemodialysis patient that are commonly associated with an arteriovenous fistula dilated venous segment in an arteriovenous fistula exhibiting high venous blood flow or/and pressure.

In some embodiments, the present invention also relates to a method for pre-determining an arteriovenous fistula reconstructed venous segment lumen diameter, whereby the pre-determined venous segment lumen diameter is to be used as guidance for treating an arteriovenous fistula dilated venous segment in a hemodialysis patient whose arteriovenous fistula exhibits abnormally high venous blood flow or/and pressure. In some embodiments, the present invention also relates to the use of an implantable blood vessel external support in the treatment of a hemodialysis arteriovenous fistula dilated venous segment.

### BACKGROUND OF THE INVENTION

In performing hemodialysis (haemodialysis, kidney dialysis, or dialysis) on a patient, a well established way of obtaining direct access to the patient's bloodstream is via an arteriovenous fistula (AVF, AV fistula, or dialysis fistula) typically located in the patient's arm. In such types of hemodialysis, ordinarily, a tube is connected to a needle inserted in a first venous entry point of the arteriovenous fistula, blood is then pumped through a blood purification device (dialyzer), and then the processed (purified) blood is pumped back into the patient's bloodstream through another tube connected to a second needle inserted in a second venous entry point of the arteriovenous fistula.

While undergoing hemodialysis, typically, the patient's blood flow properties and characteristics (e.g., blood volumetric flow rate, blood pressure, blood viscosity) are closely monitored. A generally accepted guideline is that within a typical hemodialysis access (such as an arteriovenous fistula access), normal venous blood volumetric flow rate is in the range of between about 500 milliliters per minute-1500 milliliters per minute (500 mL/min-1500 mL/min) [0.5 liter/min-1.5 liter/min], low venous blood volumetric flow rate is equal to or less than about 500 milliliters per minute (500 mL/min) [0.5 liter/min], and high venous blood volumetric flow rate is in the range of between about 1500 milliliters per minute-4000 milliliters per minute (1500 mL/min-4000 mL/min) [1.5 liter/min-4.0 liter/min].

In general, a hemodialysis type arteriovenous fistula exhibiting any abnormal venous blood volumetric flow rate is undesirable, since such can cause onset of, or/and complicate other existing, pathological conditions (particularly, as relating to the heart) in a hemodialysis patient. Hemodialysis patients having a newly formed arteriovenous fistula typically experience an immediate decrease in peripheral vascular resistance that results in a compensatory type increase in cardiac output. Over time, the arteriovenous fistula, particularly, the venous portion thereof, typically exhibits lumen dilation (e.g., via aneurysmal dilation), but, may also exhibit lumen sclerosis, where symptoms of either condition typically worsen under high blood flow rates.

Such abnormal vasculature and hemodynamic type phenomena associated with hemodialysis are usually accompanied by an undesirably large imbalance between blood flowing into, and out of, the arteriovenous fistula, for example, when the rate or/and pressure of (arterial) blood flowing into the arteriovenous fistula is substantially higher than (and possibly overwhelming) the rate or/and pressure of (venous) blood flowing out of the arteriovenous fistula. Such phenomena may result in severe dilation and swelling of the arteriovenous fistula veins (e.g., leading to a mega-fistula) in a patient's arm.

About 15-20% of hemodialysis patients have an abnormally functioning arteriovenous fistula (often accompanied by a dilated venous segment thereof), whose venous blood volumetric flow rate is greater than 2000 milliliters per minute (2000 mL/min). In some hemodialysis patients having existing heart disease, even lower, but, still relatively high, venous blood flow rates in the arteriovenous fistula can further damage the heart. For example, such an abnormally functioning arteriovenous fistula may cause chronic overload on the heart, that can lead to High Output Cardiac Failure (HOCF) in the hemodialysis patient. HOCF can result in undesirable remodeling of the heart, which, if left untreated, can become irreversible.

Although various techniques have been proposed and implemented for attempting to address and overcome the above described problems, there remains an on-going need for developing and practicing new or/and improved techniques for dealing with abnormal vasculature and hemodynamic type phenomena associated with hemodialysis, particularly, for treating an arteriovenous fistula dilated venous segment in a hemodialysis patient whose arteriovenous fistula exhibits abnormally high venous blood flow or/and pressure.

### SUMMARY OF THE INVENTION

The present invention, in some embodiments thereof, relates to treating abnormal vasculature and hemodynamic type phenomena associated with hemodialysis. More particularly, but not exclusively, the present invention relates to a method for treating an arteriovenous fistula dilated venous segment in a hemodialysis patient whose arteriovenous fistula exhibits abnormally high venous blood flow or/and pressure. The present invention is applicable for preventing or treating various pathological conditions (such as progressive heart disease, venous aneurysms, venous intimal hyperplasia, venous thrombosis, and unseemly appearance (i.e., bulges) of the arm) in a hemodialysis patient that are commonly associated with an arteriovenous fistula dilated venous segment in an arteriovenous fistula exhibiting high venous blood flow or/and pressure.

In some embodiments, the present invention also relates to a method for pre-determining an arteriovenous fistula reconstructed venous segment lumen diameter, whereby the pre-determined venous segment lumen diameter is to be used as guidance for treating an arteriovenous fistula dilated venous segment in a hemodialysis patient whose arteriovenous fistula exhibits abnormally high venous blood flow or/and pressure. In some embodiments, the present invention also relates to the use of an implantable blood vessel external support in the treatment of a hemodialysis arteriovenous fistula dilated venous segment.

According to an aspect of some embodiments of the present invention, there is provided a method for treating an arteriovenous fistula dilated venous segment in a hemodialysis patient whose arteriovenous fistula exhibits abnormally high venous blood flow or/and pressure, the method comprising: surgically reconstructing the arteriovenous fistula dilated venous segment, for forming an arteriovenous fistula reconstructed venous segment; and covering and supporting the arteriovenous fistula reconstructed venous segment with an implantable blood vessel external support, such that the arteriovenous fistula exhibits normal venous blood flow and pressure.

According to some embodiments of the invention, surgically reconstructing the arteriovenous fistula dilated venous segment includes radially cutting through an end portion of the arteriovenous fistula dilated venous segment, so as to open and disconnect the arteriovenous fistula dilated venous segment end from the adjacent remaining venous portion of the arteriovenous fistula, thereby forming an opened and disconnected end of the arteriovenous fistula dilated venous segment.

According to some embodiments of the invention, radially cutting is performed at either a proximal location or at a distal location relative to the midline of the patient's body, so as to form a corresponding proximal radial cut, or a distal radial cut, through the proximal end portion, or through the distal end portion, respectively, of the arteriovenous fistula dilated venous segment.

According to some embodiments of the invention, surgically reconstructing the arteriovenous fistula dilated venous segment includes longitudinally cutting along the arteriovenous fistula dilated venous segment, so as to form a longitudinal cut extending along the arteriovenous fistula dilated venous segment.

According to some embodiments of the invention, surgically reconstructing the arteriovenous fistula dilated venous segment includes performing a partial tissue reconstruction procedure on the arteriovenous fistula dilated venous segment, by removing venous material therefrom, so as to form the arteriovenous fistula reconstructed venous segment.

According to some embodiments of the invention, the method further comprises closing the arteriovenous fistula reconstructed venous segment, to a desired arteriovenous fistula reconstructed venous segment lumen diameter.

According to some embodiments of the invention, closing of the arteriovenous fistula reconstructed venous segment includes using a venous segment lumen diameter forming device, around which tissue of the arteriovenous fistula reconstructed venous segment is worked on, in order to facilitate forming of the desired arteriovenous fistula reconstructed venous segment lumen diameter.

According to some embodiments of the invention, the venous segment lumen diameter forming device is a mandrel or mandrel-type device.

According to some embodiments of the invention, closing of the arteriovenous fistula reconstructed venous segment includes using results obtained by performing a procedure for pre-determining the desired arteriovenous fistula reconstructed venous segment lumen diameter.

According to some embodiments of the invention, covering and supporting the arteriovenous fistula reconstructed venous segment with the implantable blood vessel external support includes using results obtained by performing a procedure for pre-determining a desired arteriovenous fistula reconstructed venous segment lumen diameter for the arteriovenous fistula reconstructed venous segment.

According to some embodiments of the invention, the implantable blood vessel external support comprises a braided tubular body having an inner diameter in a range of between 4 mm and 9 mm.

According to some embodiments of the invention, covering and supporting the arteriovenous fistula reconstructed venous segment with the implantable blood vessel external support includes performing anastomosis on the arteriovenous fistula reconstructed venous segment, by anastomotically connecting an opened and disconnected end of the arteriovenous fistula reconstructed venous segment to the adjacent remaining venous portion of the arteriovenous fistula, so as to form an anastomosis site that connects the arteriovenous fistula reconstructed venous segment to the arteriovenous fistula, such that the arteriovenous fistula reconstructed venous segment becomes a longitudinally continuous, integral part of the arteriovenous fistula.

According to some embodiments of the invention, the method further comprises making final adjustments of configuration of the blood vessel external support fitted over the arteriovenous fistula reconstructed venous segment, such that the blood vessel external support fully covers and supports entire lengths of the arteriovenous fistula reconstructed venous segment and of the anastomosis site.

According to an aspect of some embodiments of the present invention, there is provided a method for pre-determining an arteriovenous fistula reconstructed venous segment lumen diameter, whereby the pre-determined arteriovenous fistula reconstructed venous segment lumen diameter is to be used as guidance for treating an arteriovenous fistula dilated venous segment in a hemodialysis patient whose arteriovenous fistula exhibits abnormally high venous blood flow or/and pressure, the method comprising: forming a reference set of previously obtained empirical measurements of venous blood volumetric flow rate and venous lumen diameter inside the arteriovenous fistula dilated venous segment of the hemodialysis patient; and using data and information generated from a mathematical quantitative analytical model, for analyzing the reference set of previously obtained empirical measurements of the hemodialysis patient, so as to identify the pre-determined arteriovenous fistula reconstructed venous segment lumen diameter that is suitable for treating the arteriovenous fistula dilated venous segment in the hemodialysis patient.

According to some embodiments of the invention, the method further comprises: forming a reference set of previously obtained empirical measurements of mean arterial blood pressure that enters the arteriovenous fistula dilated venous segment of the hemodialysis patient; and using the reference set of the mean arterial blood pressure, for checking and confirming the identified pre-determined arteriovenous reconstructed venous segment lumen diameter as being suitable for treating the arteriovenous fistula dilated venous segment in the hemodialysis patient.

According to some embodiments of the invention, using data and information generated from the mathematical quantitative analytical model includes mathematically quantitatively analytically modelling the previously obtained vasculature and hemodynamic phenomenological empirical measurements made on hemodialysis patients treated for having arteriovenous fistulas exhibiting high venous blood flow or/and pressure.

According to some embodiments of the invention, the mathematical quantitative analytical model is used for generating the data and information in a form of values of arteriovenous fistula mean arterial blood pressure as a function of arteriovenous fistula venous segment lumen diameter, and in a form of values of arteriovenous fistula venous segment blood volumetric flow rate as a function of arteriovenous fistula venous segment lumen diameter.

According to some embodiments of the invention, the identified pre-determined arteriovenous fistula reconstructed venous segment lumen diameter is used as guidance in a procedure for surgically reconstructing the arteriovenous fistula dilated venous segment, for forming an arteriovenous fistula reconstructed venous segment in the hemodialysis patient.

According to some embodiments of the invention, the identified pre-determined arteriovenous fistula reconstructed venous segment lumen diameter is used as guidance in a procedure for selecting an inner diameter of a venous segment lumen diameter forming device, around which tissue of the arteriovenous fistula reconstructed venous segment is worked on, in order to facilitate forming of a desired arteriovenous fistula reconstructed venous segment lumen diameter.

According to some embodiments of the invention, the identified pre-determined arteriovenous fistula reconstructed venous segment lumen diameter is further used as guidance in a procedure for covering and supporting the arteriovenous fistula reconstructed venous segment with an implantable blood vessel external support, such that the arteriovenous fistula exhibits normal venous blood flow and pressure.

According to some embodiments of the invention, the identified pre-determined arteriovenous fistula reconstructed venous segment lumen diameter is used as guidance in a procedure for selecting a diameter of a blood vessel external support for covering and supporting the arteriovenous fistula reconstructed venous segment in the hemodialysis patient.

According to an aspect of some embodiments of the present invention, there is provided a use of an implantable blood vessel external support in the treatment of a hemodialysis arteriovenous fistula dilated venous segment, in a hemodialysis patient whose arteriovenous fistula exhibits abnormally high venous blood flow or/and pressure.

According to an aspect of some embodiments of the present invention, there is provided an implantable blood vessel external support for use in the treatment of a hemodialysis arteriovenous fistula dilated venous segment, in a hemodialysis patient whose arteriovenous fistula exhibits abnormally high venous blood flow or/and pressure.

All technical or/and scientific words, terms, or/and phrases, used herein have the same or similar meaning as commonly understood by one of ordinary skill in the art to which the invention pertains, unless otherwise specifically defined or stated herein. Exemplary embodiments of methods (steps, procedures), apparatuses (devices, systems, components thereof), equipment, and materials, illustratively described herein are exemplary and illustrative only and are not intended to be necessarily limiting. Although methods, apparatuses, equipment, and materials, equivalent or similar to those described herein can be used in practicing or/and testing embodiments of the invention, exemplary methods, apparatuses, equipment, and materials, are illustratively described below. In case of conflict, the patent specification, including definitions, will control.

### BRIEF DESCRIPTION OF THE SEVERAL VIEWS OF THE DRAWINGS

Some embodiments of the present invention are herein described, by way of example only, with reference to the accompanying drawings. With specific reference now to the drawings in detail, it is stressed that the particulars shown are by way of example and for purposes of illustrative description of some embodiments of the present invention. In this regard, the description taken together with the accompanying drawings make apparent to those skilled in the art how some embodiments of the present invention may be practiced.

In the drawings:

FIGS. 1A-1B are flow diagrams of an exemplary embodiment of the method (AVF dilated venous segment treating method) for treating an arteriovenous fistula dilated venous segment in a hemodialysis patient whose arteriovenous fistula exhibits abnormally high venous blood flow or/and pressure, in accordance with some embodiments of the invention;

FIGS. 2-11b are schematic views of exemplary embodiments showing implementation of sequential steps (procedures), for example, steps (procedures) presented in FIGS. 1A-1B, of the method for treating an arteriovenous fistula dilated venous segment in a hemodialysis patient whose arteriovenous fistula exhibits abnormally high venous blood flow or/and pressure, in accordance with some embodiments of the invention;

FIG. 12 is a flow diagram of an exemplary embodiment of the method (AVF reconstructed venous segment lumen diameter pre-determining method) for pre-determining an arteriovenous fistula venous segment lumen diameter suitable for an arteriovenous fistula reconstructed venous segment, whereby the method is particularly applicable to the method (of FIGS. 1A-11b) for treating an arteriovenous fistula dilated venous segment in a hemodialysis patient whose arteriovenous fistula exhibits abnormally high venous blood flow or/and pressure, in accordance with some embodiments of the invention;

FIGS. 13 and 14 are graphical presentations of exemplary calculated values of AVF mean arterial blood pressure [entering the AVF venous segment] as a function of AVF venous segment lumen diameter (for 3 different initial AVF venous segment lumen diameters of 1 cm, 1.5 cm, and 2 cm) inside a 5 cm long AVF venous segment, for different initial AVF venous segment blood volumetric flow rates of 2 liter/min (FIG. 13) and 4 liter/min (FIG. 14), respectively, generated from an analytical model that is based on previously obtained vasculature and hemodynamic phenomenological empirical measurements, of a plurality of hemodialysis patients previously treated for having AVFs exhibiting high venous blood flow or/and pressure, in accordance with some embodiments of the invention;

FIGS. 15 and 16 are graphical presentations of exemplary calculated values of AVF mean arterial blood pressure [entering the AVF venous segment] as a function of AVF venous segment lumen diameter (for 3 different initial AVF venous segment lumen diameters of 1 cm, 1.5 cm, and 2 cm) inside a 7 cm long AVF venous segment, for different initial AVF venous segment blood volumetric flow rates of 2 liter/min (FIG. 15) and 4 liter/min (FIG. 16), respectively, generated from an analytical model that is based on previously obtained vasculature and hemodynamic phenomenological empirical measurements, of a plurality of hemodialysis patients previously treated for having AVFs exhibiting high venous blood flow or/and pressure, in accordance with some embodiments of the invention.

FIGS. 17 and 18 are graphical presentations of exemplary calculated values of AVF venous segment blood volumetric flow rate as a function of AVF venous segment lumen diameter inside a 5 cm long AVF venous segment, for different initial AVF venous segment blood volumetric flow rates of 2 liter/min (FIG. 17) and 4 liter/min (FIG. 18), respectively, generated from an analytical model that is based on previously obtained vasculature and hemodynamic phenomenological empirical measurements, of a plurality of hemodialysis patients previously treated for having AVFs exhibiting high venous blood flow or/and pressure, in accordance with some embodiments of the invention; and

FIGS. 19 and 20 are graphical presentations of exemplary calculated values of AVF venous segment blood volumetric flow rate as a function of AVF venous segment lumen diameter inside a 7 cm long AVF venous segment, for different initial AVF venous segment blood volumetric flow rates of 2 liter/min (FIG. 19) and 4 liter/min (FIG. 20), respectively, generated from an analytical model that is based on previously obtained vasculature and hemodynamic phenomenological empirical measurements, of a plurality of hemodialysis patients previously treated for having AVFs exhibiting high venous blood flow or/and pressure, in accordance with some embodiments of the invention.

### DESCRIPTION OF SPECIFIC EMBODIMENTS OF THE INVENTION

The present invention, in some embodiments thereof, relates to treating abnormal vasculature and hemodynamic type phenomena associated with hemodialysis. More particularly, but not exclusively, the present invention relates to a method for treating an arteriovenous fistula dilated venous segment in a hemodialysis patient whose arteriovenous fistula exhibits abnormally high venous blood flow or/and pressure. The present invention is applicable for preventing or treating various pathological conditions (such as progressive heart disease, venous aneurysms, venous intimal hyperplasia, venous thrombosis, and unseemly appearance (i.e., bulges) of the arm) in a hemodialysis patient that are commonly associated with an arteriovenous fistula dilated venous segment in an arteriovenous fistula exhibiting high venous blood flow or/and pressure.

In some embodiments, the present invention also relates to a method for pre-determining an arteriovenous fistula reconstructed venous segment lumen diameter, whereby the pre-determined venous segment lumen diameter is to be used as guidance for treating an arteriovenous fistula dilated venous segment in a hemodialysis patient whose arteriovenous fistula exhibits abnormally high venous blood flow or/and pressure. In some embodiments, the present invention also relates to the use of an implantable blood vessel external support in the treatment of a hemodialysis arteriovenous fistula dilated venous segment.

The term ‘hemodialysis’, as used herein, in a non-limiting manner, in the context of the field and art of the herein disclosed invention, refers to a process of purifying the blood of a person whose kidneys are not normally functioning, or have entirely stopped functioning. The term ‘hemodialysis’ as used herein is also known by, synonymous with, and equivalent to, such terms as haemodialysis, kidney dialysis, and dialysis, which are also widely known and used in the field and art of the invention. For consistency, the term ‘hemodialysis’ is used throughout the present disclosure.

The term ‘arteriovenous fistula’, as used herein, in a non-limiting manner, in the context of the field and art of the herein disclosed invention, refers to the (arterial side to venous end type) arteriovenous type (anastomotic) connection between a hollow space (of an opening or lumen) in a side portion of an arterial blood vessel (artery) and a hollow space (of an opening or lumen) in an end portion of a venous blood vessel (vein). Such an arteriovenous fistula is ordinarily created for therapeutic reasons, surgically in the forearm of a hemodialysis patient, so as to facilitate direct access to the patient's bloodstream for performing hemodialysis on the patient. For better understanding of herein illustratively described exemplary embodiments of the invention, the term ‘arteriovenous fistula’, in addition to the specific singular point of connection between the arterial side portion and the venous end portion, also refers to the immediately further proximal and distal arterial and venous portions (segments) that continuously, proximally and distally extend from the specific singular point of connection between the arterial side portion and the venous end portion, respectively. The term ‘arteriovenous fistula’ as used herein is also known by, synonymous with, and equivalent to, such terms as AVF, AV fistula, and dialysis fistula, which are also widely known and used in the field and art of the invention. For consistency, the terms ‘arteriovenous fistula’ and ‘AVF’ are used throughout the present disclosure.

The term ‘segment’, as used herein, in a non-limiting manner, in the context of the field and art of the herein disclosed invention, refers to any of the (longitudinally extending) parts (pieces or portions) into which a blood vessel (particularly, a vein or artery) can be (longitudinally or lengthwise) divided along its length, whereby, such a part (piece or portion) has a generally overall tubular shape with a length (for example, a venous segment length) and a lumen diameter (for example, a venous segment lumen diameter). For example, the term ‘venous segment’ is used herein in the context of an ‘arteriovenous fistula dilated venous segment’, corresponding to a venous part (piece or portion) of an arteriovenous fistula that is dilated (enlarged) and which has a generally overall (dilated) tubular shape with a length (arteriovenous fistula dilated venous segment length) and a (dilated) lumen diameter (arteriovenous fistula dilated venous segment lumen diameter). Additionally, for example, the term ‘venous segment’ is also used herein in the context of an ‘arteriovenous fistula reconstructed venous segment’, corresponding to a venous part (piece or portion) of an arteriovenous fistula that was radially constricted (reduced), so as to become reconstructed (re-sectioned), and which has a generally overall reduced (reconstructed) tubular shape with a length (arteriovenous fistula reconstructed venous segment length) and a reduced (reconstructed) lumen diameter (arteriovenous fistula reconstructed venous segment lumen diameter).

The term ‘arteriovenous fistula dilated venous segment’, herein, abbreviated as ‘AVF dilated venous segment’, as used herein, in a non-limiting manner, in the context of the field and art of the herein disclosed invention, refers to any of the following exemplary descriptions (i), (ii), or (iii), thereof: (i) an arteriovenous fistula (AVF) venous segment whose (original, baseline, pre-treatment) lumen (i.e., inner) diameter dilated (became enlarged); (ii) an arteriovenous fistula (AVF) venous segment, located in a hemodialysis patient's arm, and is dilated (enlarged) compared to a similar arteriovenous fistula (AVF) venous segment having the exact same anatomical location in the patient's other arm; and (iii) an arteriovenous fistula (AVF) venous segment that underwent radially outward remodeling (of its lumen) due to exposure to a hemodynamic environment featuring high venous blood flow or/and pressure.

The term ‘arteriovenous fistula reconstructed venous segment’, herein, abbreviated as ‘AVF reconstructed venous segment’, as used herein, in a non-limiting manner, in the context of the field and art of the herein disclosed invention, refers to the following exemplary description thereof: an arteriovenous fistula venous segment (such as an arteriovenous fistula dilated venous segment), having an original (pre-treatment) abnormally large average lumen (inner) diameter, from which some venous material is/was surgically removed followed by closing (for example, via suturing) the venous segment, such that the resulting arteriovenous fistula venous segment becomes reconstructed (re-sectioned) and has a reconstructed (re-sectioned) average lumen diameter less than the original (pre-treatment) average lumen diameter.

The term ‘implantable blood vessel external support’, as used herein, in a non-limiting manner, in the context of the field and art of the herein disclosed invention, refers to any implantable medical device configured (shaped and sized) to be implanted and placed over an outside surface of a (natural or synthetic) bodily vessel (for example, a natural or synthetic vein or vein graft, or, of a natural or synthetic artery or artery graft) in order to externally cover and support the bodily vessel. Additionally, or alternatively, the implantable blood vessel external support is configured (shaped and sized) in order to maintain or change one or more of the bodily vessel geometrical or/and mechanical properties and durability, or/and to prevent, lessen, or/and decrease chance of failure, modification (remodeling), or/and tissue modulation (remodeling), thereof, or/and to cast or/and impose a requested shape, size, contour or/and other external boundaries of the bodily vessel. A blood vessel external support may be designed, constructed, and implemented in the form of a stent or sleeve, that includes a tubular body in the form of wires, filaments, or threads, that are braided or woven, for example, mechanically machined, and which are made from one or more of various kinds of materials or substances, such as metals, non-metals (plastics), textiles, and others. For example, for implementing exemplary embodiments of the present invention, the herein referred to and illustratively described implantable blood vessel external support may be provided from among the numerous exemplary embodiments of the external support disclosed by the same applicant/assignee of the present invention, in U.S. Patent Application Publication No. 2016/0045304 A1, and WIPO PCT Patent Application International. Publication No. WO 2012/143922 A1.

The inventor recognized that abnormal vasculature and hemodynamic phenomena (particularly, venous intimal hyperplasia, venous thrombosis, and venous aneurysmal dilation) of a hemodialysis type arteriovenous fistula are closely associated with, and the result of, high venous blood (volumetric) flow (for example, higher than about 1500 milliliters per minute (1500 mL/min)) or/and high venous blood pressure (for example, higher than about 50 mmHg) in the arteriovenous fistula. In view thereof, the inventor conceived of, developed, and implemented new and inventive techniques that provide significant, and sustainable, reduction in such high venous blood flow or/and blood pressure, which, in turn, result in effectively treating the abnormal vasculature and hemodynamic phenomena of the hemodialysis arteriovenous fistula.

Implementation of the herein disclosed techniques leads to significant and sustainable reduction in the rate of arteriovenous fistula venous blood flow to desired levels that correspond to normal venous blood flow rates (between about 500-1500 milliliters per minute (500-1500 mL/min) [0.5-1.5 liter/min]), which are needed to facilitate effective hemodialysis on a patient. Such significant and sustained rate reduction in the arteriovenous fistula venous blood flow prevents, or at least reduces (by reversing), heart overload or/and undesirable remodeling of the heart, thereby, preventing initial damage, or at least reducing progressive damage, to the heart of the hemodialysis patient. Additionally, such rate reduction in the arteriovenous fistula venous blood flow prevents, or at least reduces severity of, other pathological conditions (such as venous aneurysms, venous intimal hyperplasia, and venous thrombosis, among several possible arteriovenous fistula type diseases, as well as unseemly appearance (i.e., bulges) of the arm) in a hemodialysis patient that may be caused or accompanied by an arteriovenous fistula exhibiting high venous blood flow or/and pressure.

Accordingly, the present invention, in some embodiments thereof, also relates to a method for preventing onset of, or treating (i.e., reducing progression of), a pathological condition (such as heart disease, venous aneurysms, venous intimal hyperplasia, and venous thrombosis, among several possible arteriovenous fistula type diseases, as well as unseemly appearance (i.e., bulges) of the arm) in a hemodialysis patient having an arteriovenous fistula exhibiting high venous blood flow or/and pressure.

In exemplary embodiments, the method for treating an arteriovenous fistula dilated venous segment in a hemodialysis patient whose arteriovenous fistula exhibits abnormally high venous blood flow or/and pressure, herein, also referred to as the AVF dilated venous segment treating method, includes the following exemplary steps (procedures): surgically reconstructing the (hemodialysis) AVF dilated venous segment, for forming an AVF reconstructed venous segment; and covering and supporting the AVF reconstructed venous segment with an implantable blood vessel external support, such that the AVF will exhibit normal venous blood flow and pressure.

In exemplary embodiments, the step (procedure) of surgically reconstructing the (hemodialysis) AVF dilated venous segment includes the following exemplary steps (procedures): (a) surgically opening the patient's arm hosting the AVF, so as to expose the AVF dilated venous segment; (b) is radially cutting through an end portion of the AVF dilated venous segment, so as to open and disconnect the AVF dilated venous segment end from the adjacent remaining venous portion of the AVF, thereby forming an opened and disconnected end of the AVF dilated venous segment; (c) longitudinally cutting along the AVF dilated venous segment, so as to form a longitudinal cut extending along the AVF dilated venous segment; (d) performing a partial tissue reconstruction (re-section) procedure on the AVF dilated venous segment, by removing some venous material therefrom via the longitudinal cut, so as to form a reconstructed (re-sectioned) venous segment of the AVF reconstructed (re-sectioned) venous segment) that includes the same opened and disconnected end of the AVF dilated venous segment; and (e) closing the AVF reconstructed venous segment, along the longitudinal cut, to a desired AVF reconstructed venous segment lumen diameter.

In exemplary embodiments, the step (procedure) of covering and supporting the AVF reconstructed venous segment with an implantable blood vessel external support includes the following exemplary steps (procedures): (a) providing an implantable blood vessel external support configured (shaped and sized) for externally covering and supporting the AVF reconstructed venous segment; (b) implanting and fitting the blood vessel external support over the AVF reconstructed venous segment, such that the implanted blood vessel external support covers and supports the AVF reconstructed venous segment; (c) performing anastomosis on the AVF reconstructed venous segment, by anastomotically connecting the opened and disconnected end of the AVF reconstructed venous segment to the adjacent remaining venous portion of the AVF, so as to form an anastomosis site that connects the AVF reconstructed venous segment to the arteriovenous fistula, such that the AVF reconstructed venous segment becomes a longitudinally continuous, integral part of the AVF; (d) making final adjustments of the configuration (shape and size) of the blood vessel external support fitted over the AVF reconstructed venous segment, such that the implanted blood vessel external support fully covers and supports the entire lengths of the AVF reconstructed venous segment and of the anastomosis site; and (e) surgically closing the patient's arm within which is the arteriovenous fistula having the AVF reconstructed venous segment covered and supported by the implanted blood vessel external support.

In exemplary embodiments, the step (procedure) of closing (for example, via suturing) the AVF reconstructed venous segment, along the longitudinal cut, to a desired AVF reconstructed venous segment lumen diameter, optionally, includes use of a venous segment lumen diameter forming device (LDFD), for example, a mandrel or mandrel-type device. In exemplary embodiments, the desired AVF reconstructed venous segment lumen diameter corresponds to, or is based on, a pre-determined AVF reconstructed venous segment lumen diameter. In exemplary embodiments, the diameter of the (optionally used) venous segment lumen diameter forming device (LDFD) is selected, at least in part, based on the desired AVF reconstructed venous segment lumen diameter, and therefore, the selected diameter of the venous segment lumen diameter forming device (LDFD) also corresponds to, or is based on, the pre-determined AVF reconstructed venous segment lumen diameter.

Additionally, in exemplary embodiments, the step (procedure) of covering and supporting the AVF reconstructed venous segment with an implantable blood vessel external support includes the step (procedure) of providing the implantable blood vessel external support configured (shaped and sized) for externally covering and supporting the AVF reconstructed venous segment. In exemplary embodiments, the diameter of the blood vessel external support is also selected, at least in part, based on the desired AVF reconstructed venous segment lumen diameter to be used for closing (suturing) the AVF reconstructed venous segment, and therefore, the selected blood vessel external support diameter also corresponds to, or is based on, the pre-determined AVF reconstructed venous segment lumen diameter.

Accordingly, in exemplary embodiments, the pre-determined AVF reconstructed venous segment lumen diameter is used as guidance for: (i) forming the AVF reconstructed venous segment (for example, including optional use of a venous segment lumen diameter forming device (LDFD), for example, a mandrel or mandrel-type device), and (ii) selecting the diameter of the blood vessel external support that is to be used for covering and supporting the AVF reconstructed venous lumen segment.

In exemplary embodiments, the pre-determined AVF reconstructed venous segment lumen diameter is identified by applying a method for pre-determining an AVF reconstructed venous segment lumen diameter suitable for an AVF reconstructed venous segment, whereby, such an identified pre-determined AVF reconstructed venous segment lumen diameter is to be used as guidance for forming, and for covering, the AVF reconstructed venous segment in the hemodialysis patient to be treated for having an AVF exhibiting abnormally high venous blood flow or/and pressure.

Accordingly, in exemplary embodiments, the present invention also relates to a method for pre-determining an AVF reconstructed venous segment lumen diameter, whereby the pre-determined AVF reconstructed venous segment lumen diameter is to be used as guidance for treating an AVF dilated venous segment in a hemodialysis patient whose AVF exhibits abnormally high venous blood flow or/and pressure.

In exemplary embodiments, the method for pre-determining an AVF reconstructed venous segment lumen diameter, herein, also referred to as the AVF reconstructed venous segment lumen diameter pre-determining method, includes the following exemplary steps (procedures): forming a reference set of previously obtained empirical measurements of venous blood volumetric flow rate and venous lumen diameter inside the AVF dilated venous segment of the hemodialysis patient; and using data and information generated from a quantitative mathematical analytical model, for analyzing the reference set of previously obtained empirical measurements of the hemodialysis patient, so as to identify the pre-determined AVF reconstructed venous segment lumen diameter that is suitable for treating the AVF dilated venous segment in the hemodialysis patient.

In exemplary embodiments, the AVF reconstructed venous segment lumen diameter pre-determining method further includes forming a reference set of previously obtained empirical measurements of mean arterial blood pressure that enters the AVF dilated venous segment of the hemodialysis patient, and analyzing the reference set of the mean arterial blood pressure, for checking and confirming the identified pre-determined AVF reconstructed venous segment lumen diameter as being suitable for treating the AVF dilated venous segment in the hemodialysis patient.

In exemplary embodiments, the AVF reconstructed venous segment lumen diameter pre-determining method is particularly applicable to the AVF dilated venous segment treating method. For example, there is using the identified pre-determined AVF reconstructed venous segment lumen diameter for: (i) forming the AVF reconstructed venous segment (including optional use of a venous segment lumen diameter forming device (LDFD), for example, a mandrel or mandrel-type device), and (ii) selecting the diameter of the blood vessel external support that is to be used for covering and supporting the AVF reconstructed venous segment in the hemodialysis patient to be treated.

For purposes of further understanding exemplary embodiments of the present invention, in the following illustrative description thereof, reference is made to the drawings. Throughout the following description and accompanying drawings, same reference numbers refer to same components, elements, or features. Additionally, throughout the description the standard terms proximal, proximally, distal, and distally, are used for indicating relative anatomical locations and directions. The term proximal, as used herein, refers to a location or a direction towards or nearer to the point of reference being the midline of a patient's body, and the term distal, as used herein, refers to a location or a direction away or farther from the same point of reference being the midline of a patient's body. For clarity and consistency, these same terms also appear in the drawings.

It is to be understood that the invention is not necessarily limited in its application to any particular sequential ordering of exemplary method steps or procedures, or to particular details of construction or/and arrangement of exemplary apparatuses or devices, set forth in the following illustrative description. The invention is capable of having other exemplary embodiments, or, of being practiced or carried out in various ways.

An aspect of some embodiments of the present invention is a method for treating an arteriovenous fistula dilated venous segment in a hemodialysis patient whose arteriovenous fistula exhibits abnormally high venous blood flow or/and pressure.

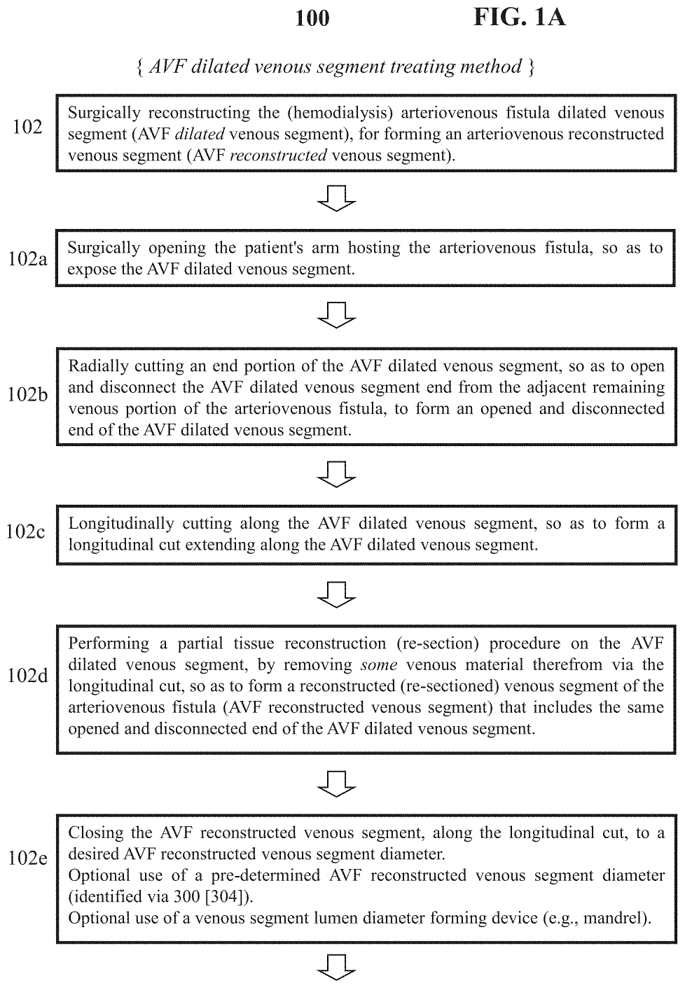

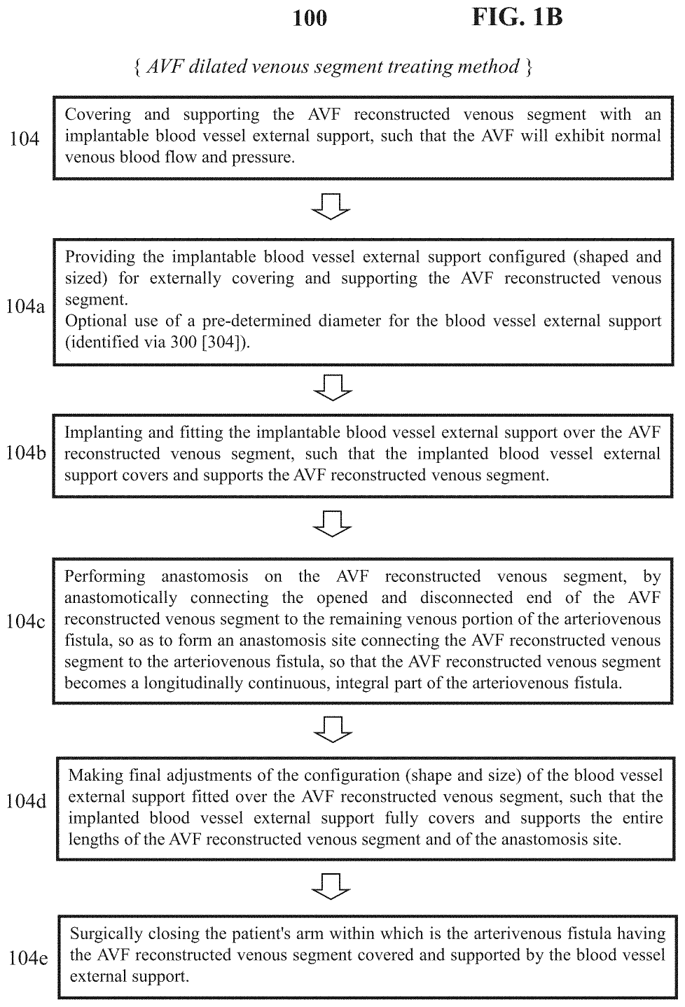

Referring now to the drawings, FIGS. 1A-1B are flow diagrams of an exemplary embodiment (indicated as, and referred to by, reference number 100), including the indicated exemplary steps (procedures, processes) thereof, of the method for treating an arteriovenous fistula dilated venous segment in a hemodialysis patient whose arteriovenous fistula exhibits abnormally high venous blood flow or/and pressure. In FIGS. 1A-1B, exemplary embodiment 100 of the AVF dilated venous segment treating method includes exemplary steps (procedures, processes) represented by separate blocks (frames) which are assigned reference numbers, for example, 102, 102a, 104, 104a, etc. For brevity, the exemplary embodiment 100 of the method for treating an arteriovenous fistula dilated venous segment in a hemodialysis patient whose arteriovenous fistula exhibits abnormally high venous blood flow or/and pressure, is also referred to as the AVF dilated venous segment treating method 100, and also as method 100.

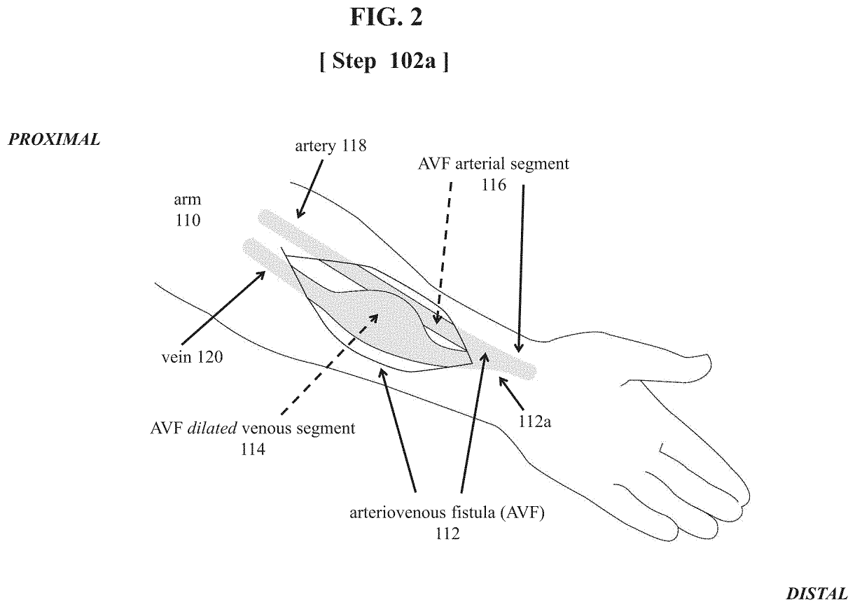

FIGS. 2-11b are schematic views of exemplary embodiments showing implementation of sequential steps (procedures), for example, steps (procedures) 102 and 104, and sub-steps thereof, presented in FIGS. 1A-1B, of the method for treating an arteriovenous fistula dilated venous segment in a hemodialysis patient whose arteriovenous fistula exhibits abnormally high venous blood flow or/and pressure.

As shown in FIGS. 1A-1B, in a non-limiting manner, and in some embodiments, such as exemplary embodiment 100, the AVF dilated venous segment treating method includes the following exemplary steps (procedures), which are also illustrated in FIGS. 2-11b.

In 102 (FIG. 1A), there is surgically reconstructing the (hemodialysis) arteriovenous fistula dilated venous segment (AVF dilated venous segment), for forming an arteriovenous fistula reconstructed venous segment (AVF reconstructed venous segment). In exemplary embodiments, step (procedure) 102 includes the following sub-steps (sub-procedures) 102a-102e.

In 102a, for example, as shown in FIG. 2, there is surgically opening the patient's arm 110 hosting the arteriovenous fistula (AVF) 112, so as to expose the arteriovenous fistula (AVF) dilated venous segment 114.

The arteriovenous fistula 112, in addition to including the specific singular point of connection 112a between the hollow space (of the opening or lumen) in the side portion of an AVF arterial segment 116 of an arterial blood vessel (artery) 118 and the hollow space (of the opening or lumen) in the distal end portion of the AVF dilated venous segment 114 of a venous blood vessel (vein) 120, also encompasses the immediately further proximal and distal arterial and venous portions (segments) that continuously, proximally and distally extend from the specific singular point of (anastomotic) connection 112a of the arteriovenous fistula 112.

The arteriovenous fistula dilated venous segment (AVF dilated venous segment) 114, in a non-limiting manner, corresponds to any of the following exemplary descriptions (i), (ii), or (iii), thereof: (i) a venous segment of the arteriovenous fistula (AVF) 112 whose (original, baseline, pre-treatment) lumen diameter dilated (became enlarged); (ii) a venous segment of the arteriovenous fistula (AVF) 112, located in the hemodialysis patient's arm 110, and is dilated (enlarged) compared to a similar arteriovenous fistula (AVF) venous segment having the exact same anatomical location in the patient's other arm; and (iii) a venous segment of the arteriovenous fistula (AVF) 112 that underwent radially outward remodeling (of its lumen) due to exposure to a hemodynamic environment featuring high venous blood flow or/and pressure.

In exemplary embodiments, step (procedure) 102a includes exposing a length of about 5 cm to 10 cm of the AVF dilated venous segment 114 proximal to the anastomotic connection 112a of the AVF 112.

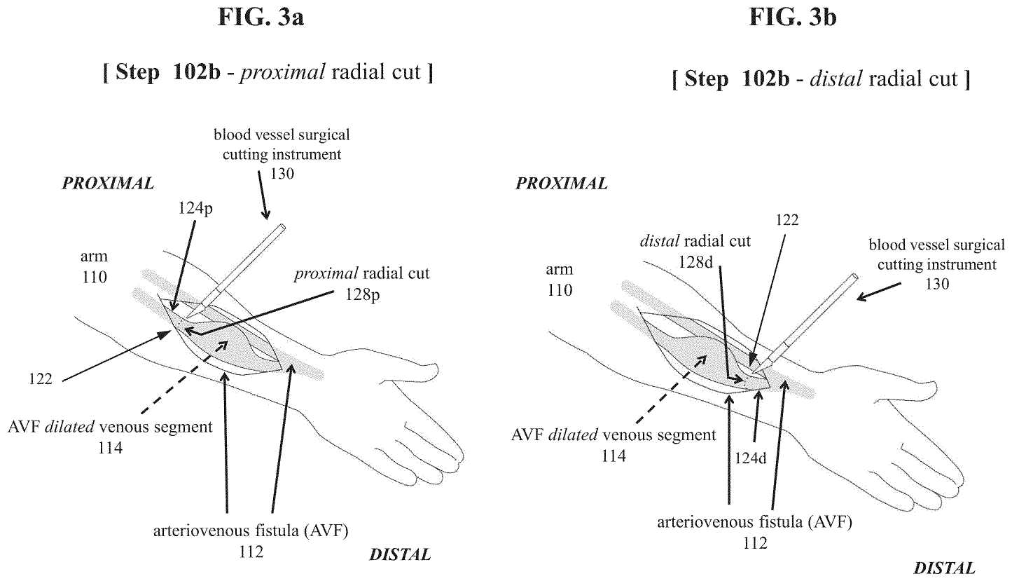

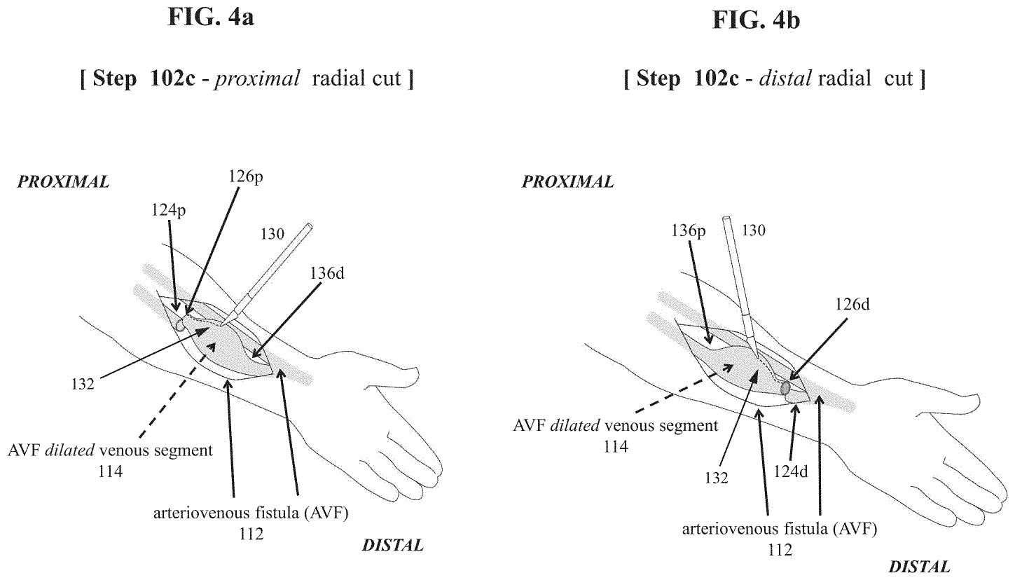

In 102b, for example, as shown in FIGS. 3a [proximal radial cut] and 3b [distal radial cut], there is radially cutting (indicated by 122) through an end portion of the AVF dilated venous segment 114, so as to open and disconnect the AVF dilated venous segment end portion from the adjacent remaining venous portion (124p or 124d) of the arteriovenous fistula 112, thereby forming an opened and disconnected end (126p or 126d, shown in FIGS. 4a and 4b, respectively) of the AVF dilated venous segment 114.

In exemplary embodiments, the radially cutting is performed at either a proximal location or at a distal location (relative to the midline of the patient's body), so as to form a corresponding proximal radial cut 128p, or a distal radial cut 128d, through the proximal end portion, or through the distal end portion, respectively, of the AVF dilated venous segment 114.

FIG. 3a [proximal radial cut] illustrates radially cutting 122 through the proximal end portion of the AVF dilated venous segment 114, so as to open and disconnect the AVF dilated venous segment proximal end portion from the adjacent remaining venous proximal portion 124p of the arteriovenous fistula 112, thereby forming an opened and disconnected proximal end (126p, FIG. 4a) of the AVF dilated venous segment 114.

FIG. 3b [distal radial cut] illustrates radially cutting 122 through the distal end of the AVF dilated venous segment 114, so as to open and disconnect the AVF dilated venous segment distal end from the adjacent remaining venous distal portion 124d of the arteriovenous fistula 112, thereby forming an opened and disconnected distal end (126d, FIG. 4b) of the AVF dilated venous segment 114.

In exemplary embodiments, radially cutting 122 through the end portion of the AVF dilated venous segment is performed using a blood vessel surgical cutting instrument 130, for example, a surgical knife, such as a scalpel or similar type of blood vessel surgical cutting instrument.

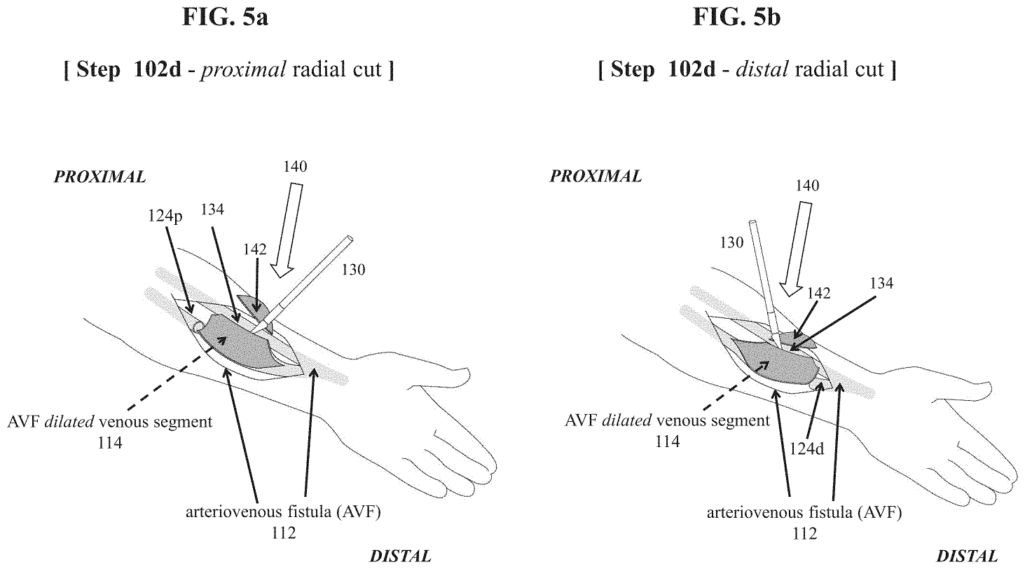

In 102c, for example, as shown in FIGS. 4a [proximal radial cut] and 4b [distal radial cut], there is longitudinally cutting (indicated by 132) along the AVF dilated venous segment 114, so as to form a longitudinal cut end (134, shown in FIGS. 5a and 5b) extending along the AVF dilated venous segment 114.

In exemplary embodiments, the longitudinally cutting 132 is performed by starting at the opened and disconnected proximal end 126p, or distal end 126d, of the AVF dilated venous segment 114, and ending at the corresponding longitudinally opposite closed distal end 136d, or proximal end 136p, of the AVF dilated venous segment 114, so as to form the longitudinal cut 134 extending along the AVF dilated venous segment 114.

FIG. 4a [proximal radial cut] illustrates longitudinally cutting 132 along the AVF dilated venous segment 114, by starting at the opened and disconnected proximal end 126p of the AVF dilated venous segment 114, and ending at the corresponding longitudinally opposite closed distal end 136d of the AVF dilated venous segment 114, so as to form a longitudinal cut 134 (FIG. 5a) extending along the AVF dilated venous segment 114.

FIG. 4b [distal radial cut] illustrates longitudinally cutting 132 along the AVF dilated venous segment 114, by starting at the opened and disconnected distal end 126d of the AVF dilated venous segment 114, and ending at the corresponding longitudinally opposite closed proximal end 136p of the AVF dilated venous segment 114, so as to form a longitudinal cut 134 (FIG. 5b) extending along the AVF dilated venous segment 114.

Although the longitudinally cutting 132 along the AVF dilated venous segment 114 is typically performed by starting at the opened and disconnected proximal end 126p or distal end 126d of the AVF dilated venous segment 114, and ending at the corresponding longitudinally opposite closed distal end 136d or proximal end 136p of the AVF dilated venous segment 114, in exemplary embodiments, the longitudinally cutting 132 is performed at various proximal or/and distal locations along the AVF dilated venous segment 114. In practice, the direction of the longitudinally cutting 132 along the AVF dilated venous segment 114 depends upon several factors, including, for example, particular patient pathology and particular morphology of the AVF 112, and of the dilated venous segment 114 thereof.

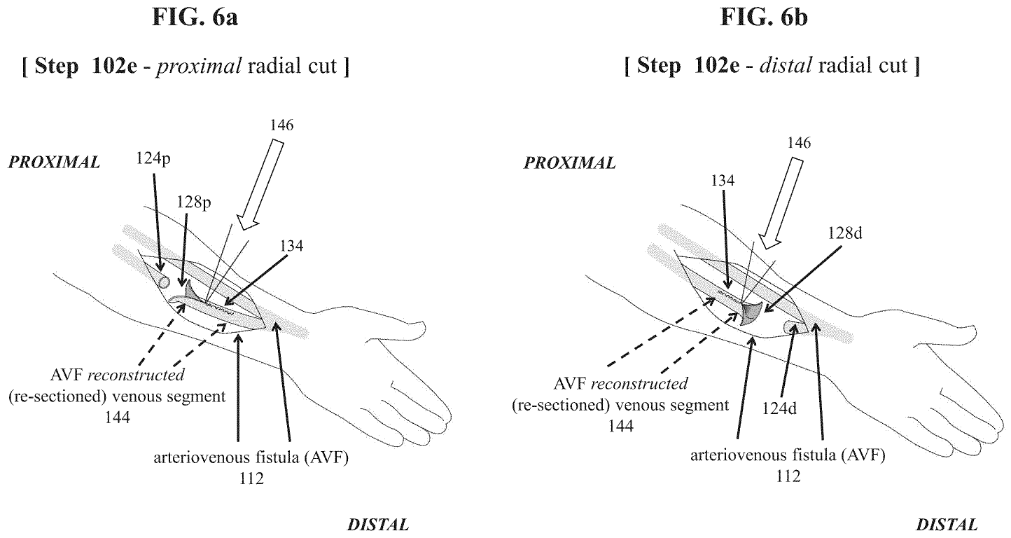

In 102d, for example, as shown in FIGS. 5a [proximal radial cut] and 5b [distal radial cut], there is performing a partial tissue reconstruction (re-section) procedure (indicated by 140) on the AVF dilated venous segment 114, by removing some venous material 142 therefrom via the longitudinal cut 134, so as to form a reconstructed (re-sectioned) venous segment (144, shown in FIGS. 6a and 6b) of the arteriovenous fistula 112 (AVF reconstructed (re-sectioned) venous segment 144) that includes the same opened and disconnected end of the AVF dilated venous segment 114.

FIG. 5a [proximal radial cut] illustrates performing the partial tissue reconstruction (re-section) procedure 140 on the AVF dilated venous segment 114 (having the proximal radial cut 128p), by removing some venous material 142 therefrom via the longitudinal cut 134, so as to form a reconstructed (re-sectioned) venous segment (144, FIG. 6a) of the arteriovenous fistula 112 (AVF reconstructed (re-sectioned) venous segment 144) that includes the same opened and disconnected proximal end 126p of the AVF dilated venous segment 114.

FIG. 5b [distal radial cut] illustrates performing the partial tissue reconstruction (re-section) procedure 140 on the AVF dilated venous segment 114 (having the distal radial cut 128d), by removing some venous material 142 therefrom via the longitudinal cut 134, so as to form a reconstructed (re-sectioned) venous segment (144, FIG. 6b) of the arteriovenous fistula 112 (AVF reconstructed (re-sectioned) venous segment 144) that includes the same opened and disconnected distal end 126d of the AVF dilated venous segment 114.

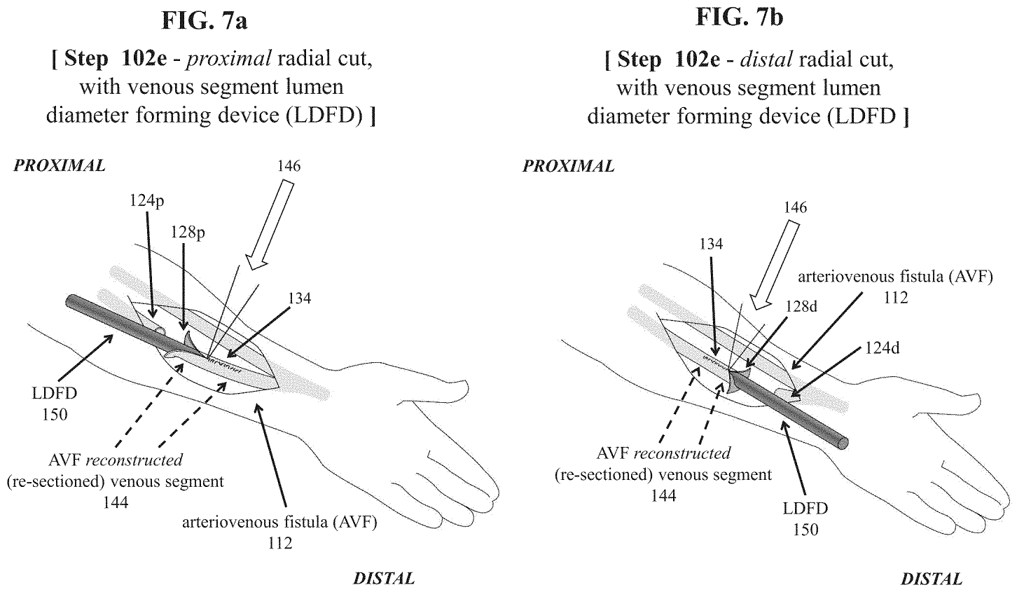

In 102e, for example, as shown in FIGS. 6a [proximal radial cut] and 6b [distal radial cut], and FIGS. 7a [proximal radial cut, with venous segment lumen diameter forming device (LDFD)] and 7b [distal radial cut, with venous segment lumen diameter forming device (LDFD)], there is closing (for example, via suturing) (indicated by 146) the AVF reconstructed venous segment 144, along the longitudinal cut 134, to a desired AVF reconstructed venous segment lumen diameter (i.e., the AVF reconstructed venous segment inner diameter not including the wall thickness of the AVF reconstructed venous segment).

The arteriovenous fistula reconstructed venous segment (AVF reconstructed venous segment) 144, in a non-limiting manner, corresponds to the following exemplary description thereof: a venous segment of the arteriovenous fistula 112 (such as AVF dilated venous segment 114), having an original (pre-treatment) abnormally large average lumen diameter, from which some venous material (for example, 142, FIGS. 5a, 5b) is/was surgically removed followed by closing (for example, via suturing) 146 the venous segment, such that the resulting venous segment of the arteriovenous fistula 112 becomes reconstructed (re-sectioned) and has a reconstructed (re-sectioned) average lumen diameter less than the original (pre-treatment) average lumen diameter.

FIG. 6a [proximal radial cut] illustrates closing (for example, via suturing) 146 the AVF reconstructed venous segment 144 (having the proximal radial cut 128p), along the longitudinal cut 134, to a desired AVF reconstructed venous segment lumen diameter.

FIG. 6b [distal radial cut] illustrates closing (for example, via suturing) 146 the AVF reconstructed venous segment 144 (having the distal radial cut 128d), along the longitudinal cut 134, to a desired AVF reconstructed venous segment lumen diameter.

In exemplary embodiments, step (procedure) 102e of closing (for example, via suturing) 146 the AVF reconstructed venous segment 144, along the longitudinal cut 134, to a desired AVF reconstructed venous segment lumen diameter, includes use of a venous segment lumen diameter forming device 150, herein, also referred to as LDFD 150, for example, a mandrel or mandrel-type device, around which tissue along the longitudinal cut 134 of the AVF reconstructed venous segment is worked on (manipulated, shaped) during the closing (for example, suturing) procedure, in order to facilitate forming of the desired AVF reconstructed venous segment lumen diameter of the AVF reconstructed venous segment.

FIG. 7a [proximal radial cut, with venous segment lumen diameter forming device (LDFD)] illustrates closing (for example, via suturing) 146 the AVF reconstructed venous segment 144 (having the proximal radial cut 128p), over (upon) the appropriately inserted and positioned venous segment lumen diameter forming device (LDFD) 150, and along the longitudinal cut 134, to a desired AVF reconstructed venous segment lumen diameter.

FIG. 7b [distal radial cut, with venous segment lumen diameter forming device (LDFD)] illustrates closing (for example, via suturing) 146 the AVF reconstructed venous segment 144 (having the distal radial cut 128d), over (upon) the appropriately inserted and positioned venous segment lumen diameter forming device (LDFD) 150, and along the longitudinal cut 134, to a desired AVF reconstructed venous segment lumen diameter.

In exemplary embodiments involving use of the venous segment lumen diameter forming device (LDFD) 150, closing (for example, suturing) 146 of the AVF reconstructed venous segment 144 is performed by accurately and uniformly constricting (reducing) the lumen diameter over, and along, the length of the venous segment lumen diameter forming device (LDFD) 150, so as to form the AVF reconstructed venous segment 144 having the desired AVF reconstructed venous segment lumen diameter being highly uniform (i.e., the same) along the entire length of the AVF reconstructed venous segment 144. Accordingly, the venous segment lumen diameter forming device (LDFD) is used in order to facilitate formation of a highly accurate and uniform reduced (reconstructed) lumen diameter along the entire length of the AVF reconstructed venous segment 144.

In exemplary embodiments, the venous segment lumen diameter forming device (LDFD) 150 is a mandrel or mandrel-type device, configured as an entirely or partly solid or hollow, rod, bar, or shaft, particularly suitable for being placed inside the lumen of the (not yet closed) AVF reconstructed venous segment 144, and around which tissue along the longitudinal cut 134 of the AVF reconstructed venous segment is worked on (manipulated, shaped) during the closing (for example, suturing) procedure, in order to facilitate formation of a uniform reduced (reconstructed) lumen diameter along the entire length of the AVF reconstructed venous segment 144.

In exemplary embodiments, the venous segment lumen diameter forming device (LDFD) 150 is made of surgical grade metal or/and non-metal (plastic) materials. In exemplary embodiments, the venous segment lumen diameter forming device (LDFD) 150 has a length in the range of between about 10 cm and 30 cm, where the length is sufficiently long for manually holding onto and manipulating by an operator, along with being sufficiently long for readily inserting the venous segment lumen diameter forming device (LDFD) into, and along, the lumen of the AVF reconstructed venous segment 144 that will be worked on (manipulated, shaped) during the closing (for example, suturing) procedure.

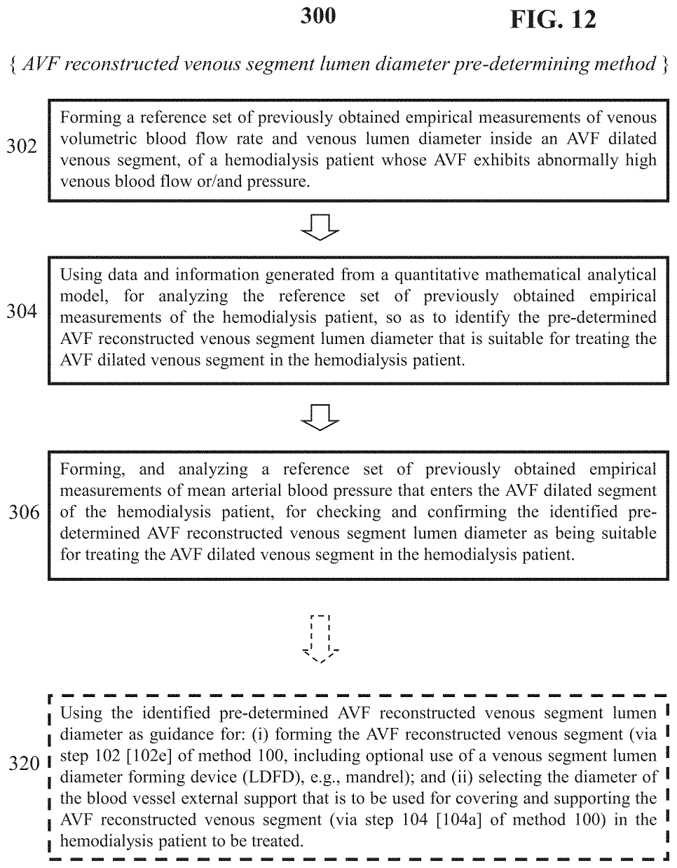

In exemplary embodiments, the venous segment lumen diameter forming device (LDFD) 150 has an outer diameter in the range of between about 4 mm (0.4 cm) and 8 mm (0.8 cm), with a typically employed range of between about 4 mm (0.4 cm) and 6 mm (0.6 cm). Such an outer diameter of the venous segment lumen diameter forming device (LDFD) corresponds to the pre-determined AVF reconstructed venous segment lumen diameter identified by applying the hereinbelow illustratively described method (for example, method 300, FIG. 12) for pre-determining an AVF reconstructed venous segment lumen diameter, whereby the pre-determined AVF reconstructed venous segment lumen diameter is to be used as guidance for treating an AVF dilated venous segment in a hemodialysis patient whose AVF exhibits abnormally high venous blood flow or/and pressure.

In exemplary embodiments, step (procedure) 102e of closing (for example, via suturing) 146 the AVF reconstructed venous segment 144, along the longitudinal cut 134, to a desired AVF reconstructed venous segment lumen diameter [either without or with use of the venous segment lumen diameter forming device (LDFD) 150], includes using results obtained by performing the hereinbelow illustratively described method (for example, method 300, FIG. 12) for pre-determining an AVF reconstructed venous segment lumen diameter.

For example, as shown in FIG. 12, in step (procedure) 320, there is using the identified pre-determined AVF reconstructed venous segment lumen diameter [from 304, 306] as guidance for forming the AVF reconstructed venous segment 144 in the hemodialysis patient being treated.

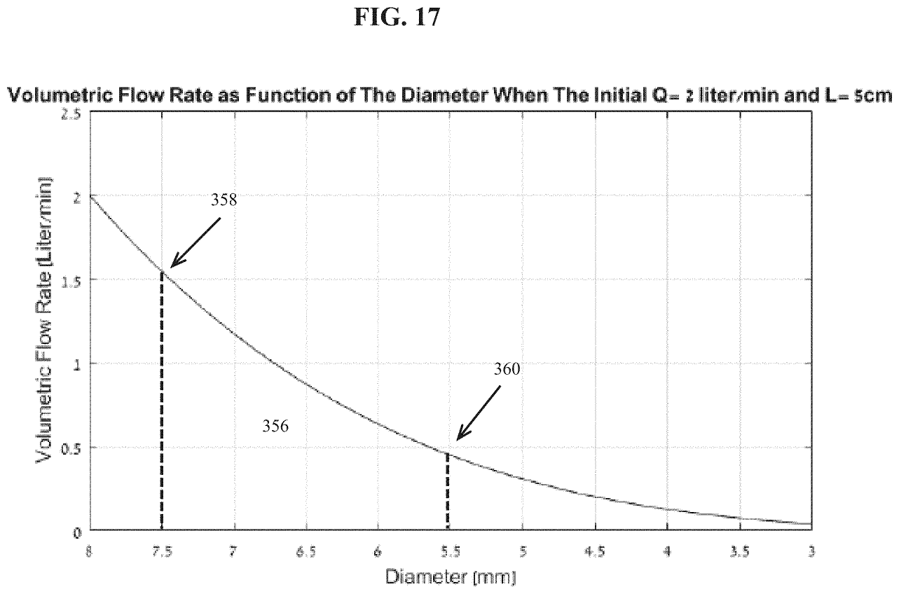

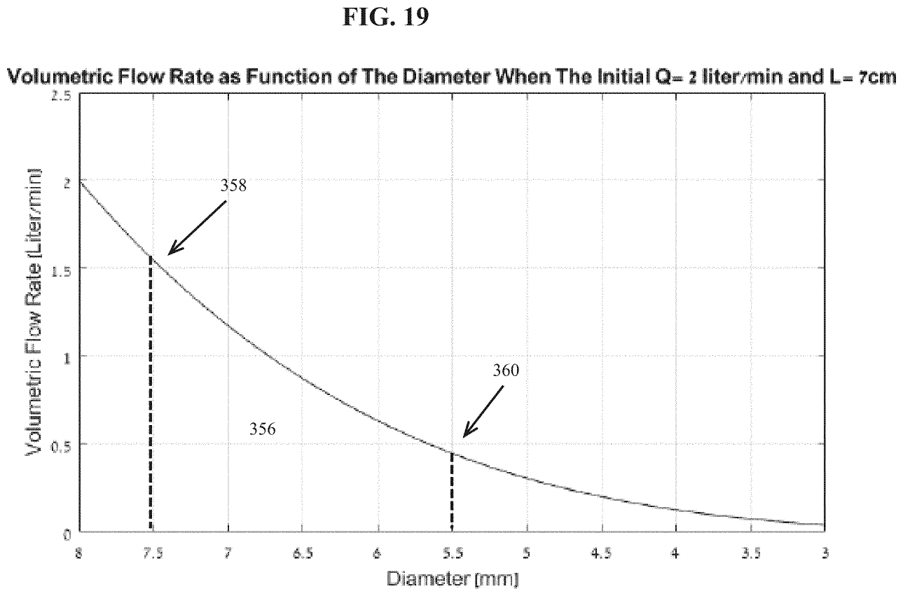

Specifically, for example, in a first exemplary scenario, wherein the initial venous segment blood volumetric flow rate inside the patient's AVF dilated venous segment 114 is equal to, or about (close to), 2 liter/min, using and analyzing the data and information generated from the hereinbelow described quantitative mathematical analytical model (for example, as presented in FIGS. 17 and 19), leads to identifying the pre-determined AVF reconstructed venous segment lumen diameter as being in the range of between about 7.5 mm (0.75 cm) and about 5.5 mm (0.55 cm), that is suitable for reconstructing (constricting and reducing) the lumen diameter of the AVF dilated venous segment 114 in the hemodialysis patient being treated.

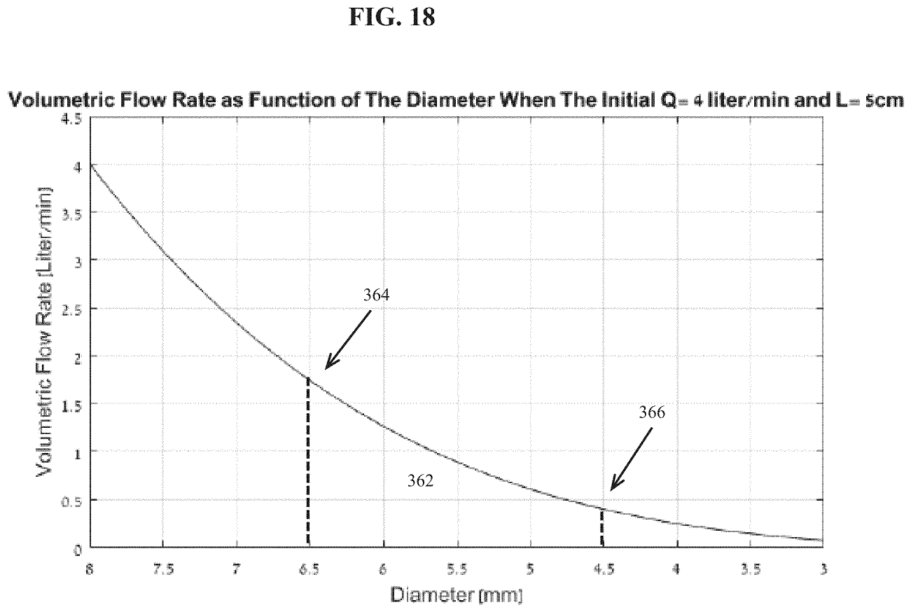

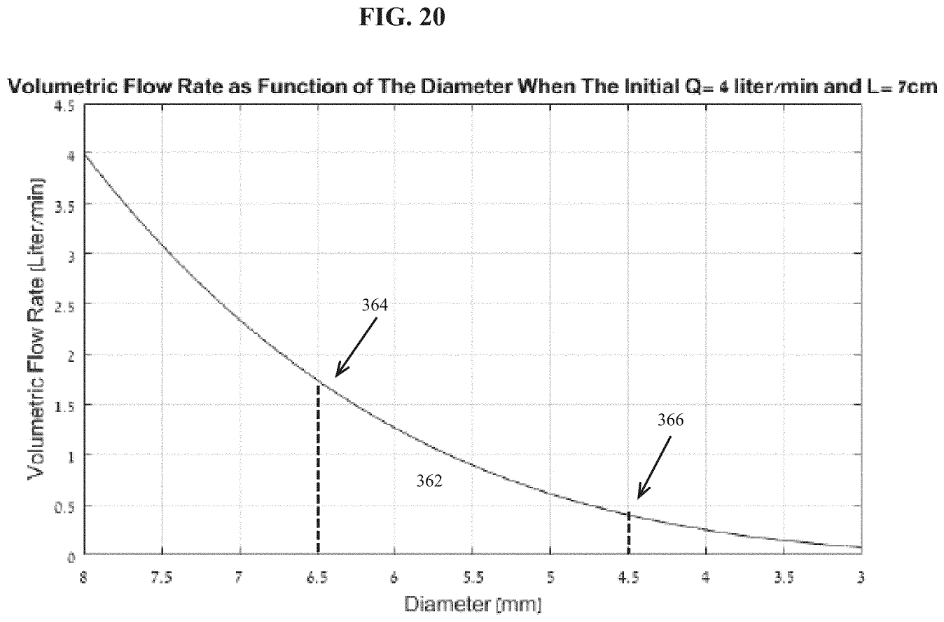

Specifically, for example, in a second exemplary scenario, wherein the initial venous segment blood volumetric flow rate inside the patient's AVF dilated venous segment being equal to, or about (close to), 4 liter/min, using and analyzing the data and information generated from the hereinabove described quantitative mathematical analytical model (for example, as presented in FIGS. 18 and 20), leads to identifying the pre-determined AVF reconstructed venous segment lumen diameter as being in the range of between about 6.5 mm (0.65 cm) and about 4.5 mm (0.45 cm), that is suitable for reconstructing (constricting and reducing) the lumen diameter of the AVF dilated venous segment 114 in the hemodialysis patient being treated.

Then, according to step (procedure) 306 (FIG. 12), there is forming a reference set of previously obtained empirical measurements of mean arterial blood pressure that enters the AVF dilated venous segment 114 of the hemodialysis patient, and analyzing the reference set of the mean arterial blood pressure, for checking and confirming the identified pre-determined AVF reconstructed venous segment lumen diameter as being suitable for reconstructing (constricting and reducing) the lumen diameter of the AVF dilated venous segment 114 in the hemodialysis patient being treated.

In 104 (FIG. 1B), there is covering and supporting the AVF reconstructed venous segment with an implantable blood vessel external support, such that the arteriovenous fistula will exhibit normal venous blood flow and pressure. In exemplary embodiments, step (procedure) 104 includes the following sub-steps (sub-procedures) 104a-104e.

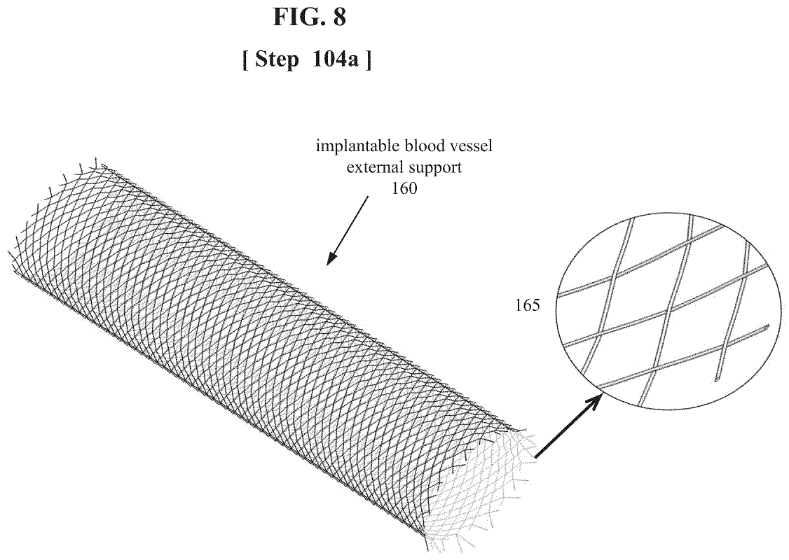

In 104a, for example, as shown in FIG. 8, there is providing an implantable blood vessel external support, for example, blood vessel external support 160, configured (shaped and sized) for externally covering and supporting the AVF reconstructed venous segment 144.

The implantable blood vessel external support 160, in a non-limiting manner, is any implantable medical device configured (shaped and sized) to be implanted and placed over an outside surface of a (natural or synthetic) bodily vessel (for example, a natural or synthetic vein or vein graft, or, of a natural or synthetic artery or artery graft), for example, of the AVF reconstructed venous segment 144, in order to externally cover and support the bodily vessel, for example, AVF reconstructed venous segment 144.

Additionally, or alternatively, the implantable blood vessel external support 160 is configured (shaped and sized) to maintain or change one or more of the AVF reconstructed venous segment 144 geometrical and/or mechanical properties and durability, or/and to prevent, lessen, or/and decrease chance of failure, modification (remodeling), or/and tissue modulation (remodeling), thereof, or/and to cast or/and impose a requested shape, size, contour or/and other external boundaries of thereof.

The implantable blood vessel external support 160 is designed, constructed, and implemented in the form of a stent or sleeve, that includes a tubular body in the form of wires, filaments, or threads, that are braided or woven, for example, mechanically machined, and which are made from one or more of various kinds of materials or substances, such as metals, non-metals (plastics), textiles, and others. For example, for implementing exemplary embodiments of the present invention, the herein referred to and illustratively described implantable blood vessel external support 160 may be provided from among the numerous exemplary embodiments of the external support disclosed by the same applicant/assignee of the present invention, in U.S. Patent Application Publication No. 2016/0045304 A1, and WIPO PCT Patent Application International Publication No. WO 2012/143922 A1.

In exemplary embodiments, the provided blood vessel external support 160 is configured to have one or more of the following possible exemplary structural properties, characteristics, and ranges of size dimensions:
braided, with wires (filaments), for example, in FIG. 8, indicated by the close-up view 165.
woven.
number of wires (filaments): in a range of between 20 and 70, for example, 42.
wire (filament) diameter: in a range of between 30 microns and 90 microns, for example, 50 microns.
braiding angle between the wires (filaments): in a range of between 90 degrees and 130 degrees.
inner diameter: in a range of between 4 mm and 9 mm.
(longitudinal) length: in a range of between 2 cm (20 mm) and 15 cm (150 mm).
material(s) of construction: metallic, for example, stainless steel, nitinol, cobalt chrome, or, non-metallic, for example, Teflon® (polytetrafluorethylene), Dacron® (synthetic polyester fiber).
elastic, and not plastically deformable.
porous or fenestrated, with pores or windows, for example, in FIG. 8, indicated by the close-up view 165.

In exemplary embodiments, the blood vessel external support 160 has an inner diameter that is selected (for example, by an operator prior to, or during, treatment of the AVF dilated venous segment 114 in a hemodialysis patient) according to the following procedure:
(1) identifying a physiologically desired (suitable, acceptable) AVF reconstructed venous segment lumen diameter [either without or with use of the venous segment lumen diameter forming device (LDFD) 150], for performing step 102 [102e] (FIGS. 1A, 6a, 6b, 7a, 7b) of surgically reconstructing the AVF dilated venous segment 114, for forming the AVF reconstructed venous segment 144.
(2) calculating the inner diameter of the blood vessel external support based on: (i) the identified desired AVF reconstructed venous segment lumen diameter, and (ii) an average wall thickness of 0.5 mm for the AVF reconstructed venous segment 144. Thus, the inner diameter of the blood vessel external support equals the sum of the value of (i) plus twice the value of (ii), namely, (i)+2x(ii), or (i)+2x(0.5 mm), or (i)+1.0 mm.

This is the value of the inner diameter of the blood vessel external support that is to be selected for implanting, and then, covering and supporting, the AVF reconstructed venous segment 144 in the hemodialysis patient.

As illustratively described hereinabove, in exemplary embodiments, step (procedure) 102e of closing (for example, via suturing) 146 the AVF reconstructed venous segment 144, along the longitudinal cut 134, to a desired AVF reconstructed venous segment lumen diameter [either without or with use of the venous segment lumen diameter forming device (LDFD) 150], includes using results obtained by performing the hereinbelow illustratively described method (for example, method 300, FIG. 12) for pre-determining an AVF reconstructed venous segment lumen diameter. For example, as shown in FIG. 12, in step (procedure) 320, there is using the identified pre-determined AVF reconstructed venous segment lumen diameter [from 304, 306] as guidance for selecting the diameter of the blood vessel external support that is to be used for covering and supporting the AVF reconstructed venous segment (via step 104 [104a] of method 100) in the hemodialysis patient to be treated.

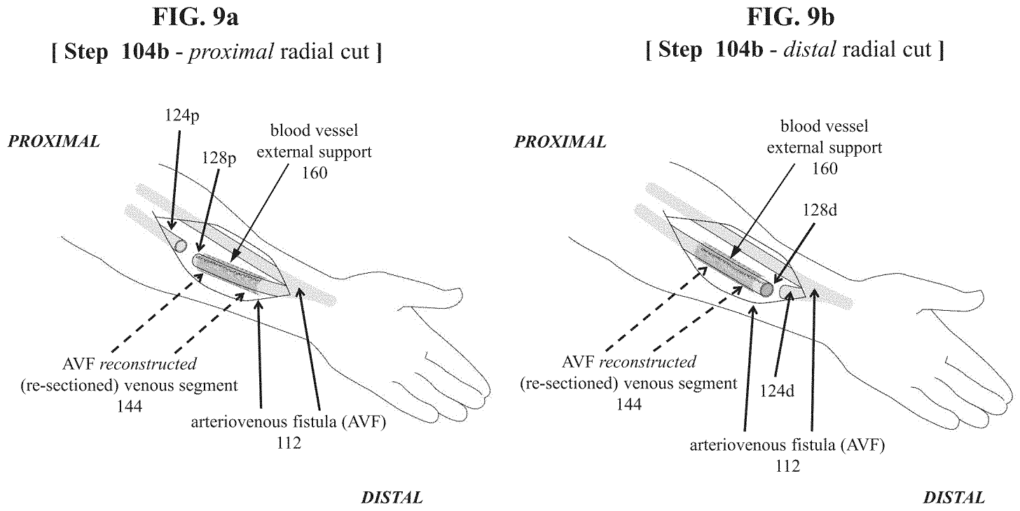

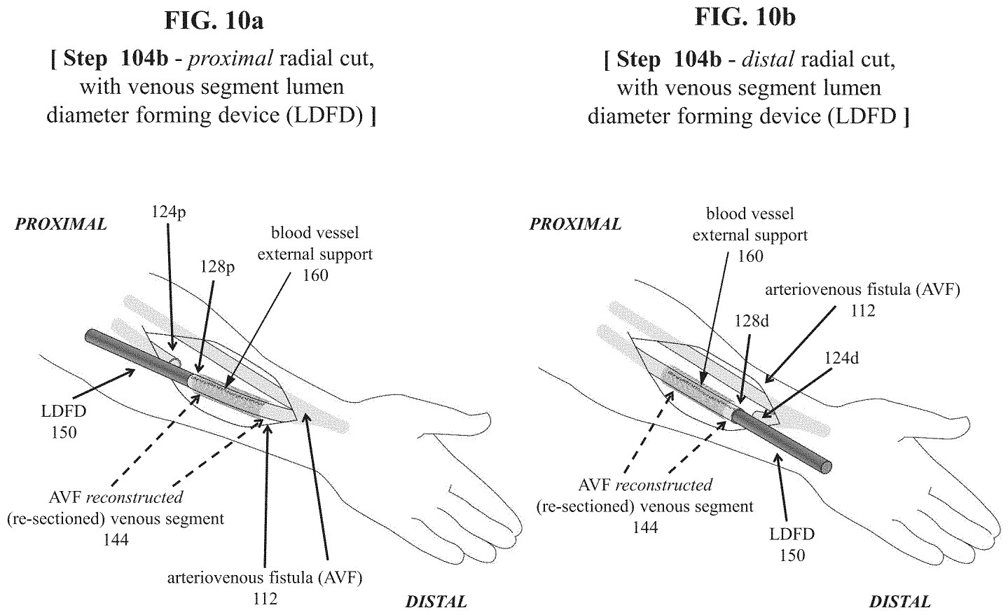

In 104b, for example, as shown in FIGS. 9a [proximal radial cut] and 9b [distal radial cut], and FIGS. 10a [proximal radial cut, with venous segment lumen diameter forming device (LDFD)] and 10b [distal radial cut, with venous segment lumen diameter forming device (LDFD)], there is implanting and fitting the blood vessel external support 160 over the AVF reconstructed venous segment 144, such that the implanted blood vessel external support 160 covers and supports the AVF reconstructed venous segment 144.

FIG. 9a [proximal radial cut] illustrates implanting and fitting the blood vessel external support 160, via the proximal radial cut 128p, over the AVF reconstructed venous segment 144, such that the implanted blood vessel external support 160 covers and supports the AVF reconstructed venous segment 144.

FIG. 9b [distal radial cut] illustrates implanting and fitting the blood vessel external support 160, via the distal radial cut 128d, over the AVF reconstructed venous segment 144, such that the implanted blood vessel external support 160 covers and supports the AVF reconstructed venous segment 144.

In exemplary embodiments, fitting of the blood vessel external support 160 over the AVF reconstructed venous segment 144 is facilitated by radially and longitudinally configuring (expanding, contracting) the blood vessel external support 160 to a desired diameter and length.

FIG. 10a [proximal radial cut, with venous segment lumen diameter forming device (LDFD)] illustrates implanting and fitting the blood vessel external support 160 (previously placed on, and then distally slid off of, the venous segment lumen diameter forming device (LDFD) 150), via the proximal radial cut 128p, over the AVF reconstructed venous segment 144, such that the implanted blood vessel external support 160 covers and supports the AVF reconstructed venous segment 144.

FIG. 10b [distal radial cut, with venous segment lumen diameter forming device (LDFD)] illustrates implanting and fitting the blood vessel external support 160 (previously placed on, and then proximally slid off of, the venous segment lumen diameter forming device (LDFD) 150), via the distal radial cut 128d, over the AVF reconstructed venous segment 144, such that the implanted blood vessel external support 160 covers and supports the AVF reconstructed venous segment 144.

In exemplary embodiments involving use of the mandrel 150, the blood vessel external support 160 is threaded over the venous segment lumen diameter forming device (LDFD) 150, followed by radially and longitudinally configuring (expanding, contracting) the blood vessel external support 160 to a desired diameter and length.

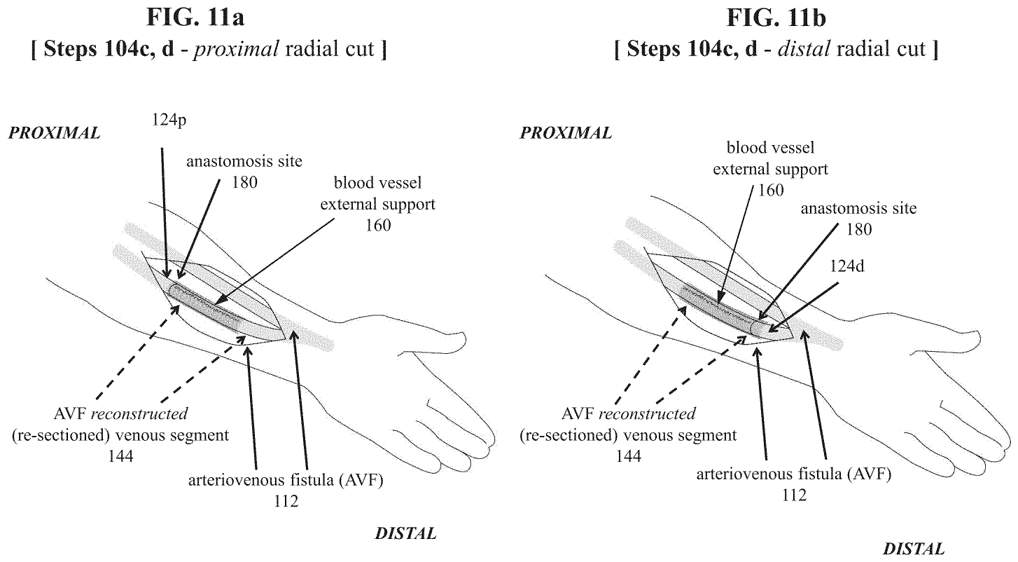

In 104c, for example, as shown in FIGS. 11a [proximal radial cut] and 11b [distal radial cut], there is performing anastomosis on the AVF reconstructed venous segment 144, by anastomotically connecting the opened and disconnected end (126p or 126d), via the radial cut (128p or 128d, respectively), of the AVF reconstructed venous segment 144 to the adjacent remaining venous portion (124p or 124d, respectively) of the arteriovenous fistula (AVF) 112, so as to form a (vein end to vein end) anastomosis site 180 that connects the AVF reconstructed venous segment 144 to the arteriovenous fistula (AVF) 112, such that the AVF reconstructed venous segment 144 becomes a longitudinally continuous, integral part of the arteriovenous fistula (AVF) 112.

FIG. 11a [proximal radial cut] illustrates performing anastomosis on the AVF reconstructed venous segment 144, by anastomotically connecting the opened and disconnected proximal end 126p, via the proximal radial cut 128p, of the AVF reconstructed venous segment 144 to the adjacent remaining venous proximal portion 124p of the arteriovenous fistula (AVF) 112, so as to form a (vein end to vein end) anastomosis site 180 that connects the AVF reconstructed venous segment 144 to the arteriovenous fistula (AVF) 112, such that the AVF reconstructed venous segment 144 becomes a longitudinally continuous, integral part of the arteriovenous fistula (AVF) 112.

FIG. 11b [distal radial cut] illustrates performing anastomosis on the AVF reconstructed venous segment 144, by anastomotically connecting the opened and disconnected distal end 126d, via the distal radial cut 128d, of the AVF reconstructed venous segment 144 to the adjacent remaining venous distal portion 124d of the arteriovenous fistula (AVF) 112, so as to form a (vein end to vein end) anastomosis site 180 that connects the AVF reconstructed venous segment 144 to the arteriovenous fistula (AVF) 112, such that the AVF reconstructed venous segment 144 becomes a longitudinally continuous, integral part of the arteriovenous fistula (AVF) 112.

In 104d, for example, also as shown in FIGS. 11a [proximal radial cut] and 11b [distal radial cut], there is making final adjustments of the configuration (shape and size) of the implanted blood vessel external support 160 fitted over the AVF reconstructed venous segment 144, such that the implanted blood vessel external support 160 fully covers and supports the entire lengths of the AVF reconstructed venous segment 144 and of the anastomosis site 180.

In 104e, there is surgically closing the patient's arm 110 within which is the arteriovenous fistula (AVF) 112 having the AVF reconstructed venous segment 114 covered and supported by the blood vessel external support 160.

As described hereinabove, in exemplary embodiments, the AVF dilated venous segment treating method, for example, exemplary embodiment 100, includes the step (procedure) 102 (FIG. 1A) of surgically reconstructing the (hemodialysis) AVF dilated venous segment, for forming an AVF reconstructed venous segment. In exemplary embodiments, the step (procedure) 102 includes the step (procedure) 102e (FIGS. 1A, 6a, 6b, 7a, 7b), of closing (for example, suturing) the AVF reconstructed venous segment, along the longitudinal cut, to a desired AVF reconstructed venous segment lumen diameter, which, in turn, optionally includes use of a venous segment lumen diameter forming device (LDFD), for example, a mandrel or mandrel-type device. In exemplary embodiments, the desired AVF reconstructed venous segment lumen diameter corresponds to, or is based on, a pre-determined AVF reconstructed venous segment lumen diameter. In exemplary embodiments, the diameter of the (optionally used) venous segment lumen diameter forming device (LDFD) is selected, at least in part, based on the desired AVF reconstructed venous segment lumen diameter, and therefore, the selected diameter of the venous segment lumen diameter forming device (LDFD) also corresponds to, or is based on, the pre-determined AVF reconstructed venous segment lumen diameter.

Additionally, in exemplary embodiments, the AVF dilated venous segment treating method 100 includes the step (procedure) 104 (FIG. 1B) of covering and supporting the AVF reconstructed venous segment with an implantable blood vessel external support, such that the AVF will exhibit normal venous blood flow and pressure. In exemplary embodiments, the step (procedure) 104 includes the step (procedure) 104a (FIGS. 1B, 8), of providing the implantable blood vessel external support configured (shaped and sized) for externally covering and supporting the AVF reconstructed venous segment. In exemplary embodiments, the diameter of the blood vessel external support is also selected, at least in part, based on the desired AVF reconstructed venous segment lumen diameter to be used for closing (for example, via suturing) the AVF reconstructed venous segment along the longitudinal cut, and therefore, the selected blood vessel external support diameter also corresponds to, or is based on, the pre-determined AVF reconstructed venous segment lumen diameter.

Accordingly, in exemplary embodiments, the pre-determined AVF reconstructed venous segment lumen diameter is used as guidance for: (i) forming the AVF reconstructed venous segment (for example, via step 102 [102e] of method 100, including optional use of a venous segment lumen diameter forming device (LDFD)), and (ii) selecting the diameter of the blood vessel external support that is to be used for covering and supporting the AVF reconstructed venous lumen segment (for example, via step 104 [104a] of method 100).

In exemplary embodiments, the pre-determined AVF reconstructed venous segment lumen diameter is identified by applying a method for pre-determining an arteriovenous fistula reconstructed venous segment lumen diameter suitable for an AVF reconstructed venous segment, whereby, the identified pre-determined AVF reconstructed venous segment lumen diameter is to be used as guidance for forming, and, for covering and supporting, the AVF reconstructed venous segment in the hemodialysis patient to be treated for having an arteriovenous fistula exhibiting abnormally high venous blood flow or/and pressure.

Accordingly, another aspect of some embodiments of the present invention is a method for pre-determining an arteriovenous fistula reconstructed venous segment lumen diameter, whereby the pre-determined arteriovenous fistula reconstructed venous segment lumen diameter is to be used as guidance for treating an arteriovenous fistula dilated venous segment in a hemodialysis patient whose arteriovenous fistula exhibits abnormally high venous blood flow or/and pressure.

FIG. 12 is a flow diagram of an exemplary embodiment (indicated as, and referred to by, reference number 300), including the indicated exemplary steps (procedures, processes) thereof, of the method for pre-determining an arteriovenous fistula reconstructed venous segment lumen diameter, whereby the pre-determined arteriovenous fistula reconstructed venous segment lumen diameter is to be used as guidance for treating an arteriovenous fistula dilated venous segment in a hemodialysis patient whose arteriovenous fistula exhibits abnormally high venous blood flow or/and pressure. For brevity, the exemplary embodiment 300 of the method for pre-determining an arteriovenous fistula reconstructed venous segment lumen diameter suitable for an arteriovenous fistula reconstructed venous segment, is also referred to as the AVF reconstructed venous segment lumen diameter pre-determining method 300, and also as method 300.

As shown in FIG. 12, in a non-limiting manner, and in some embodiments, such as exemplary embodiment 300, the AVF reconstructed venous segment lumen diameter pre-determining method includes the following exemplary steps (procedures).

In 302, there is forming a reference set of previously obtained empirical measurements of venous blood volumetric flow rate and venous lumen diameter inside the arteriovenous fistula dilated venous segment of the hemodialysis patient.

In 304, there is using data and information generated from a quantitative mathematical analytical model, for analyzing the reference set of previously obtained empirical measurements of the hemodialysis patient, so as to identify the pre-determined arteriovenous fistula reconstructed venous segment lumen diameter that is suitable for treating the arteriovenous fistula dilated venous segment in the hemodialysis patient.

In exemplary embodiments, the AVF reconstructed venous segment lumen diameter pre-determining method 300 further includes the step (procedure) 306. In 306, there is forming a reference set of previously obtained empirical measurements of mean arterial blood pressure that enters the arteriovenous fistula dilated venous segment of the hemodialysis patient, and analyzing the reference set of the mean arterial blood pressure, for checking and confirming the identified pre-determined arteriovenous reconstructed venous segment lumen diameter as being suitable for treating the arteriovenous fistula dilated venous segment in the hemodialysis patient.

In exemplary embodiments, the AVF reconstructed venous segment lumen diameter pre-determining method 300 is particularly applicable to the method (for example, method 100 presented in FIGS. 1A-11b) for treating an arteriovenous fistula dilated venous segment in a hemodialysis patient whose arteriovenous fistula exhibits abnormally high venous blood flow or/and pressure. For example, as shown in FIG. 12, in 320 (indicated by the dashed arrow and box), there is using the identified pre-determined AVF reconstructed venous segment lumen diameter [from 304, 306] for: (i) forming the AVF reconstructed venous segment (via step 102 [102e] of method 100, including optional use of a venous segment lumen diameter forming device (LDFD)); and (ii) selecting the diameter of the blood vessel external support that is to be used for covering and supporting the AVF reconstructed venous segment (via step 104 [104a] of method 100) in the hemodialysis patient to be treated.

As part of developing and implementing (practicing) exemplary embodiments of the herein disclosed method for treating an arteriovenous dilated venous segment in a hemodialysis patient whose arteriovenous fistula exhibits abnormally high venous blood flow or/and pressure, for example, as illustratively described herein (with reference to FIGS. 1A-11b), the inventor reviewed, analyzed, and evaluated vast amounts of vasculature and hemodynamic phenomenological empirical measurements (parametric data and information) that were previously recorded from actual clinical studies performed on numerous hemodialysis patients already treated for having arteriovenous fistulas exhibiting abnormally high venous blood flow or/and pressure (typically, where the arteriovenous fistula included at least one arteriovenous fistula dilated venous segment). Exemplary vasculature and hemodynamic phenomenological empirical measurements (parametric data and information) that were previously recorded from such actual clinical studies, and which were reviewed, analyzed, and evaluated, by the inventor, include the following:
AVF mean arterial blood pressure [entering the AVF venous segment] as a function of AVF venous segment lumen diameter
   For different initial AVF venous segment lumen diameters, inside different lengths of an AVF venous segment (uncovered and absent a blood vessel external support), for different initial AVF venous segment blood volumetric flow rates (inside the AVF venous segments).
   AVF mean arterial blood pressure is the pressure inside the AVF arterial segment immediately next to the anastomosis site with the AVF venous segment, corresponding to the AVF mean arterial blood pressure entering the AVF venous segment. AVF mean arterial blood pressure data are based on measurements made using pressure sensors located inside the AVF arterial segment. AVF venous segment lengths, and external (i.e., not lumen) diameters, were measured using rulers during surgery.
   Exemplary different initial AVF venous segment lumen diameters were: 1 cm, 1.5 cm, and 2 cm.
   Exemplary different AVF venous segment lengths were: 5 cm and 7 cm.
   Exemplary different initial AVF venous segment blood volumetric flow rates were: 2 liter/min and 4 liter/min.
AVF venous segment blood volumetric flow rate as a function of AVF venous segment lumen diameter
   For inside different lengths of an AVF venous segment (uncovered and absent a blood vessel external support), for different initial AVF venous segment blood volumetric flow rates (inside the AVF venous segments).
   AVF venous segment blood volumetric flow rate data are based on calculations made from measurements of AVF venous and arterial vessel lumen diameters and blood velocities using ultrasound techniques (for example, Doppler type ultrasound). AVF venous segment lengths, and external (i.e., not lumen) diameters, were measured using rulers during surgery.
   Exemplary different AVF venous segment lengths were: 5 cm and 7 cm.
   Exemplary different initial AVF venous segment blood volumetric flow rates were: 2 liter/min and 4 liter/min.

The above previously obtained vasculature and hemodynamic phenomenological empirical measurements (parametric data and information) were used for forming various exemplary reference sets of previously obtained empirical measurements of the AVF mean arterial blood pressure as a function of AVF venous segment lumen diameter, and of the AVF venous segment blood volumetric flow rate as a function of AVF venous segment lumen diameter.

During review, analysis, and evaluation of the previously obtained vasculature and hemodynamic phenomenological empirical measurements (parametric data and information), the inventor noticed several (qualitative and quantitative) trends therein, and interpreted such trends in the context of knowing that abnormal vasculature and hemodynamic phenomena (particularly, venous intimal hyperplasia, venous thrombosis, and venous aneurysmal dilation) of a hemodialysis type arteriovenous fistula are closely associated with, and the result of, high venous blood (volumetric) flow (for example, higher than about 1500 milliliters per minute (1500 mL/min)) or/and high venous blood pressure (for example, higher than about 50 mmHg) in the arteriovenous fistula. In view thereof, the inventor focused on developing a technique having as at least one objective to provide significant, and sustainable, reduction in such high AVF venous blood flow or/and blood pressure, which, in turn, would result in effectively treating the abnormal vasculature and hemodynamic phenomena of the hemodialysis arteriovenous fistula (particularly, of an AVF dilated venous segment thereof).

As part of attaining such an objective, the inventor conceived of, developed, and implemented, a mathematical quantitative analytical model that is used for mathematically quantitatively analytically modelling the previously obtained vasculature and hemodynamic phenomenological empirical measurements (parametric data and information) made on numerous hemodialysis patients treated for having arteriovenous fistulas exhibiting high venous blood flow or/and pressure.

In exemplary embodiments, the analytical model is based on a form of Poiseuille's Law of fluid dynamics, which relates volume flow of an incompressible fluid through a tube (hollow cylinder), namely, the volume flow of an incompressible fluid through a tube is equal to it/8 times the pressure differences between the ends of the tube, times the fourth power of the tube's radius divided by the product of the tube's length and the dynamic viscosity of the fluid. An exemplary mathematical form of Poiseuille's Law is as follows:

$$Poiseuille ' s Law \text{:} Q = \frac{π Pr4}{8 η L}$$

where:
Q is the volumetric flow rate of the incompressible fluid flowing through the tube.
π is the universal constant (whose value is 3.14) of the ratio of the circumference of a circle to its diameter.
P is the pressure difference between the ends of the tube.
r is the radius of the tube (not including the wall thickness of the tube).
η is the dynamic viscosity of the fluid.
L is the length of the tube.

According to exemplary embodiments of the analytical model developed and implemented herein, for mathematically quantitatively analytically modelling the previously obtained vasculature and hemodynamic phenomenological empirical measurements (parametric data and information) made on numerous hemodialysis patients treated for having arteriovenous fistulas exhibiting high venous blood flow or/and pressure, the following corresponding parameters of Poiseuille's Law are applicable:
Q is the volumetric flow rate of blood flowing through an AVF venous segment lumen. Exemplary units are liters/minute (L/min), or milliliters/minute (mL/min).
P is the AVF mean arterial blood pressure inside the AVF arterial segment immediately next to the anastomosis site with the AVF venous segment, corresponding to the AVF meant arterial blood pressure entering the AVF venous segment. Exemplary units are mmHg.
r is the radius of the AVF venous segment lumen (i.e., not including the wall thickness of the AVF venous segment). Exemplary units are centimeters (cm) or millimeters (mm) for the diameter of the AVF venous segment lumen.
η is the dynamic viscosity of the blood flowing through the AVF venous segment lumen.
L is the length of the AVF venous segment. Exemplary units are centimeters (cm).

In exemplary embodiments, the analytical model was used for mathematically quantitatively analytically modelling the exemplary reference sets of previously obtained empirical measurements of the AVF mean arterial blood pressure as a function of AVF venous segment lumen diameter, and of the AVF venous segment blood volumetric flow rate as a function of AVF venous segment lumen diameter. For example, the analytical model was used for generating data and information in the form of values of AVF mean arterial blood pressure as a function of AVF venous segment lumen diameter, and in the form of values of AVF venous segment blood volumetric flow rate as a function of AVF venous segment lumen diameter, whereby such generated data and information (values) were graphically presented for additional review, analysis, and evaluation. Such data and information that were generated from the mathematical quantitative analytical model were applied to the herein disclosed AVF reconstructed venous segment lumen diameter pre-determining method, for example, method 300 shown in FIG. 12.

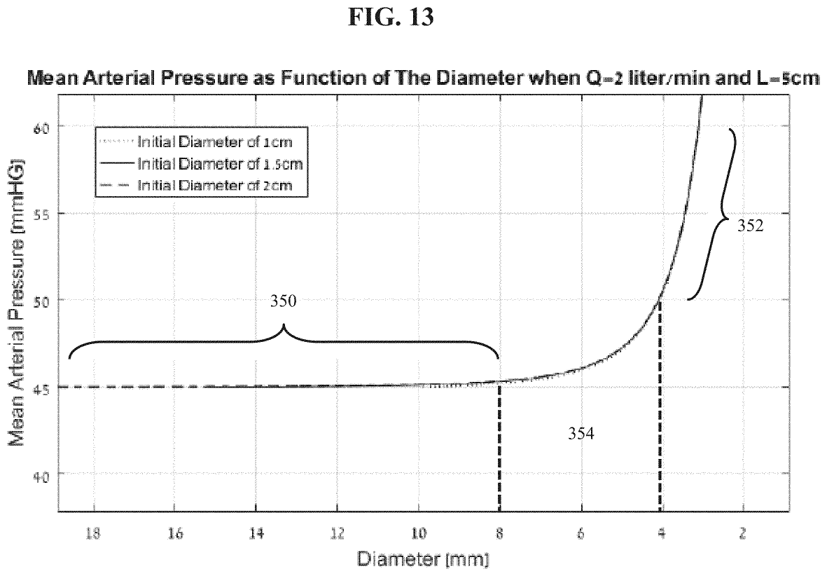

Specific examples of using the analytical model for generating values of AVF mean arterial blood pressure as a function of AVF venous segment lumen diameter, and for generating values of AVF venous segment blood volumetric flow rate as a function of AVF venous segment lumen diameter, and then graphically presenting such generated values, are shown in FIGS. 13-20.

As a first step in developing and implementing the mathematical quantitative analytical model, there was calculating pressure differences for high flow AVFs (typically, having at least one AVF dilated venous segment), for a range of AVF venous segment blood volumetric flow rates (for example, 2 liters/min to 5 liters/min), AVF venous segment lengths (for example, 5 cm and 7 cm), and initial AVF venous segment lumen diameters (for example, 1 cm, 1.5 cm, 2 cm, and 2.5 cm). Based on having previously obtained values of the AVF mean arterial pressure near the AVF anastomosis (i.e., near the arteriovenous type (anastomotic) connection between the hollow space (of the opening or lumen) in the side portion of the AVF arterial blood vessel (artery) and the hollow space (of the opening or lumen) in the end portion of the AVF venous blood vessel (vein), an objective was to calculate nominal values for the AVF venous pressure (P) at the end of the AVF venous segment (thereby, simulating pressure entering and inside an AVF reconstructed venous segment). Such values of AVF venous pressure are different for (i.e., a function of): (i) AVF venous segment length (corresponding to the length of the AVF reconstructed venous segment to be covered and supported by a blood vessel external support), (ii) initial AVF venous segment lumen diameter, and (iii) initial AVF venous segment blood volumetric flow rate. Values for (i), (ii), and (iii) were known from the previously obtained vasculature and hemodynamic phenomenological empirical measurements (parametric data and information) made on numerous hemodialysis patients treated for having arteriovenous fistulas exhibiting high venous blood flow or/and pressure.

As a second step in developing and implementing the mathematical quantitative analytical model, there was analytically (mathematically, quantitatively) simulating (imitating) an exemplary (real, clinically based type) scenario of treating an AVF dilated venous segment in a hemodialysis patient whose AVF exhibits high venous blood flow or/and pressure. According to such an exemplary simulated scenario, for a given (fixed or constant) AVF venous segment length (for example, 5 cm or 7 cm), and for a given (fixed or constant) initial AVF venous segment blood volumetric flow rate (for example, 2 liter/min or 4 liter/min), and for different initial AVF venous segment lumen diameters (for example, 1 cm, 1.5 cm, or 2 cm), there was simulating gradual constricting (reducing) of the AVF venous segment lumen diameter, in order to simulate the affect on the AVF mean arterial pressure [entering the AVF venous segment].

Ordinarily, it is expected that as the AVF venous segment (dilated) lumen diameter is constricted (reduced), the AVF mean arterial pressure [entering the AVF venous segment] will likewise increase, as a compensatory type effect, in order to produce sufficient pressure gradient to maintain the relatively high blood volumetric flow rate through the AVF venous segment (i.e., high AVF venous segment blood volumetric flow rate). The results of the analytical (mathematical, quantitative) simulations (imitations) of such an exemplary (real, clinically based type) scenario are graphically presented in FIGS. 13-16.

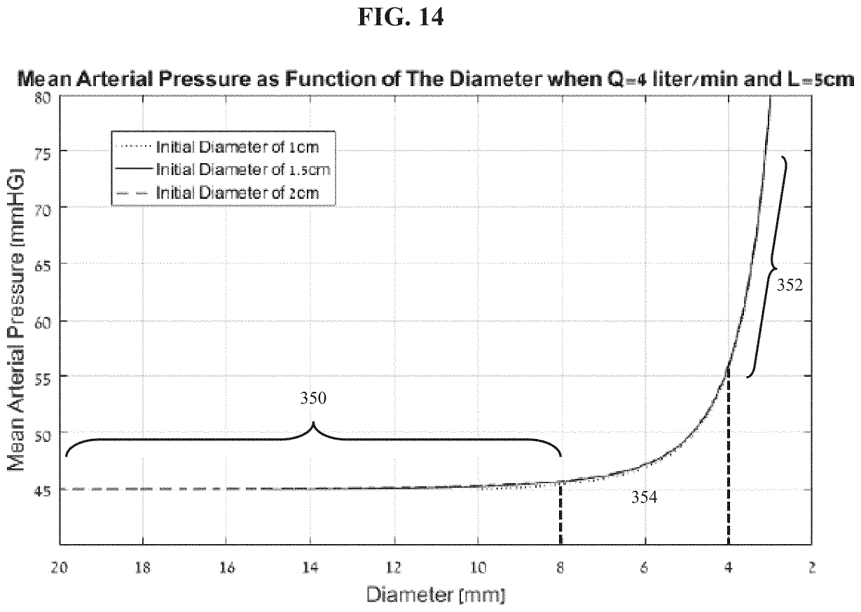

FIGS. 13 and 14 are graphical presentations of exemplary calculated values of AVF mean arterial blood pressure [entering the AVF venous segment] as a function of AVF venous segment lumen diameter (for 3 different initial AVF venous segment lumen diameters of 1 cm, 1.5 cm, and 2 cm) inside a 5 cm long AVF venous segment, for different initial AVF venous segment blood volumetric flow rates of 2 liter/min (FIG. 13) and 4 liter/min (FIG. 14), respectively, generated from an analytical model that is based on previously obtained vasculature and hemodynamic phenomenological empirical measurements, of a plurality of hemodialysis patients previously treated for having AVFs exhibiting high venous blood flow or/and pressure.

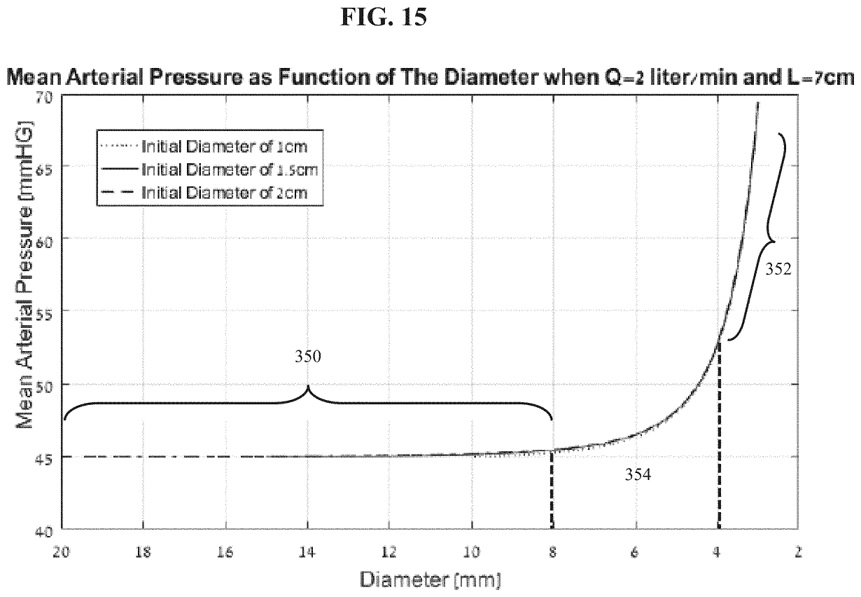

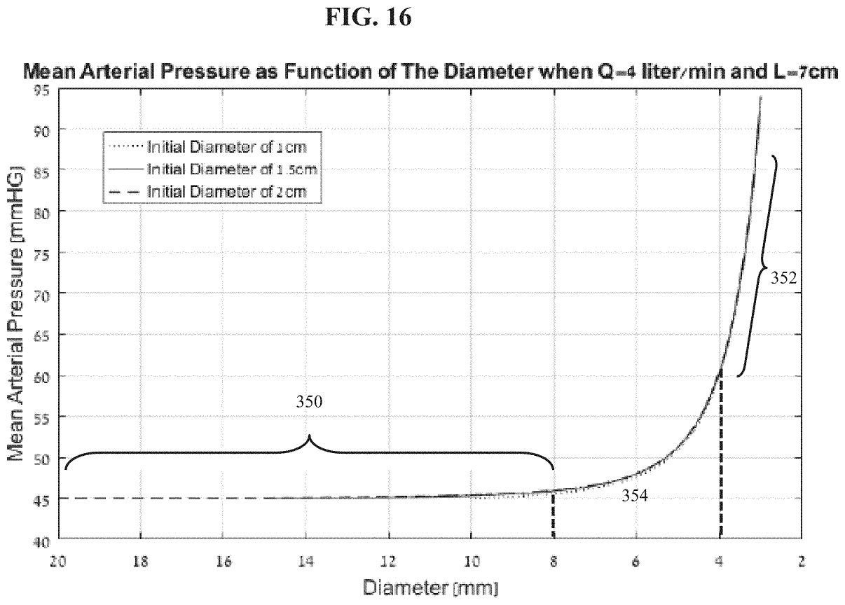

FIGS. 15 and 16 are graphical presentations of exemplary calculated values of AVF mean arterial blood pressure [entering the AVF venous segment] as a function of AVF venous segment lumen diameter (for 3 different initial AVF venous segment lumen diameters of 1 cm, 1.5 cm, and 2 cm) inside a 7 cm long AVF venous segment, for different initial AVF venous segment blood volumetric flow rates of 2 liter/min (FIG. 15) and 4 liter/min (FIG. 16), respectively, generated from an analytical model that is based on previously obtained vasculature and hemodynamic phenomenological empirical measurements, of a plurality of hemodialysis patients previously treated for having AVFs exhibiting high venous blood flow or/and pressure.

Highlights and main findings of, and guidance provided by, the data and information presented in FIGS. 13-16 are as follows:
1) Reduction in the AVF venous segment lumen diameter from an initial diameter of 1 cm (10 mm), 1.5 cm (15 mm), and 2 cm (20 mm), down to a reduced (reconstructed) diameter of about 0.8 cm (8 mm), results in a negligible increase in AVF mean arterial blood pressure [entering the AVF venous segment], as indicated in FIGS. 13-16 by the parenthesized portion 350.
   This finding leads to the guidance that for implementing the AVF dilated venous segment treating method (for example, method 100, as illustratively described herein with reference to FIGS. 1A-11b), particularly, step (procedure) 102 [102d, 102e], the AVF dilated venous segment lumen diameter should be reduced (reconstructed, re-sectioned) such that the resulting AVF reconstructed venous segment lumen diameter will be less than about 0.8 cm (8 mm), without causing undesirable significant increase in AVF mean arterial blood pressure [entering the AVF reconstructed venous segment] in the hemodialysis patient.
2) Reduction in the AVF venous segment lumen diameter from an initial diameter of 1 cm (10 mm), 1.5 cm (15 mm), and 2 cm (20 mm), down to a reduced (reconstructed) diameter of less than about 0.4 cm (4 mm), results in a relatively large increase in AVF mean arterial blood pressure [entering the AVF venous segment], as indicated in FIGS. 13-16 by the parenthesized portion 352.
   Such a relatively large increase in AVF mean arterial blood pressure causes an increase in peripheral resistance in and along the AVF venous segment, leading to an increase in systemic blood pressure and cardiac load, which is an undesirable effect in hemodialysis patients having AVFs. This finding leads to the guidance that for implementing the AVF dilated venous segment treating method (for example, method 100, as illustratively described herein with reference to FIGS. 1A-11b), particularly, step (procedure) 102 [102d, 102e], the AVF dilated venous segment lumen diameter should be reduced (reconstructed, re-sectioned) such that the AVF reconstructed venous segment lumen diameter will be greater than about 0.4 cm (4 mm), in order to prevent undesirable large increase in AVF mean arterial blood pressure [entering the AVF reconstructed venous segment] in the hemodialysis patient.
3) The results of (1) and (2) are essentially independent of the initial AVF venous segment lumen diameter, at least, in the range of between 2 cm (20 mm) and 1 cm (10 mm).
4) The results of (1) and (2) are essentially independent of the initial AVF venous segment blood volumetric flow rate, at least, in the range of between 2 liter/min and 4 liter/min.
5) The results of (1) and (2) are essentially independent of the AVF venous segment length, at least, in the range of between 5 cm and 7 cm.

The above findings lead to the guidance that the most suitable reduced (reconstructed) AVF venous segment lumen diameter is in the range of between about 0.8 cm (8 mm) and about 0.4 cm (4 mm), as indicated in FIGS. 13-16 by the dashed lines portion 354. This guidance is based on the findings that: (1) reduced (reconstructed) AVF venous segment lumen diameters down to about 0.8 cm (8 mm) will not cause undesirable significant increase in AVF mean arterial blood pressure [entering the AVF reconstructed venous segment] in the hemodialysis patient, and (2) reduced (reconstructed) AVF venous segment lumen diameters less than about 0.4 cm (4 mm) will cause undesirable large increase in AVF mean arterial blood pressure [entering the AVF reconstructed venous segment] in the hemodialysis patient.

The above findings were very unexpected, and, in fact, counter-intuitive, regarding the nature of the functional relationship and dependence of AVF mean arterial blood pressure [entering the AVF venous segment] on the AVF venous segment lumen diameter, for the following reasons.

First, it was intuitively expected that the AVF mean arterial blood pressure [entering the AVF venous segment] would start to meaningfully increase with AVF venous segment lumen diameter, throughout the ‘entire’ range of reduction in AVF venous segment lumen diameter, and ‘not only’ for AVF lumen diameters less than about 0.8 cm (8 mm).

Second, it was also intuitively expected that such relatively large increase in the AVF mean arterial blood pressure for AVF venous segment lumen diameters less than about 0.4 cm (4 mm) would be at least ‘somewhat’ dependent upon the initial AVF venous segment lumen diameter (in the range of between 2 cm (20 mm) and 1 cm (10 mm)), instead of being essentially independent thereupon.

Third, it was also expected that the functional relationship and dependence of AVF mean arterial blood pressure [entering the AVF venous segment] on the AVF venous segment lumen diameter, would be at least ‘somewhat’ dependent upon the initial AVF venous segment blood volumetric flow rate (in the range of between 2 liter/min and 4 liter/min) or/and dependent upon the AVF venous segment length (in the range of between 5 cm and 7 cm), instead of being essentially independent upon each of these additional vasculature and hemodynamic phenomenological parameters.

As a third step in developing and implementing the mathematical quantitative analytical model, there was assuming that systemic arterial pressure is regulated and balanced, and therefore, once the pressure in the arterial side of the AVF increases above the regulated and balanced mean arterial pressure at steady state, blood is redistributed to areas with less peripheral resistance. Based on such assumption, the mean arterial pressure [entering the AVF venous segment] was considered constant, in order to find out how constriction (reduction) of the AVF venous segment lumen diameter, particularly, for reduced (reconstructed) AVF venous segment lumen diameters in the range of between about 0.8 cm (8 mm) and about 0.4 cm (4 mm), affects the AVF venous segment blood volumetric flow rate, for different AVF venous segment lengths, and for different initial AVF venous segment blood volumetric flow rates.

Accordingly, there was additionally analytically (mathematically, quantitatively) simulating (imitating) the exemplary (real, clinically based type) scenario of treating an AVF dilated venous segment in a hemodialysis patient whose AVF exhibits high venous blood flow or/and pressure, as follows. For a given (fixed or constant) AVF venous segment length (for example, 5 cm or 7 cm), and for a given (fixed or constant) initial AVF venous segment blood volumetric flow rate (for example, 2 liter/min or 4 liter/min), and for a given (fixed) AVF mean arterial pressure (for example, 40-70 mmHg), there was simulating gradual constricting (reducing) of the AVF venous segment lumen diameter, particularly, for reduced (reconstructed) AVF venous segment lumen diameter is in the range of between about 0.8 cm (8 mm) and about 0.4 cm (4 mm), in order to simulate the affect thereof on the AVF venous segment blood volumetric flow rate. The results of the additional analytical (mathematical, quantitative) simulations (imitations) of such an exemplary (real, clinically based type) scenario are graphically presented in FIGS. 17-20.

FIGS. 17 and 18 are graphical presentations of exemplary calculated values of AVF venous segment blood volumetric flow rate as a function of AVF venous segment lumen diameter inside a 5 cm long AVF venous segment, for different initial AVF venous segment blood volumetric flow rates of 2 liter/min (FIG. 17) and 4 liter/min (FIG. 18), respectively, generated from an analytical model that is based on previously obtained vasculature and hemodynamic phenomenological empirical measurements, of a plurality of hemodialysis patients previously treated for having AVFs exhibiting high venous blood flow or/and pressure.

FIGS. 19 and 20 are graphical presentations of exemplary calculated values of AVF venous segment blood volumetric flow rate as a function of AVF venous segment lumen diameter inside a 7 cm long AVF venous segment, for different initial AVF venous segment blood volumetric flow rates of 2 liter/min (FIG. 19) and 4 liter/min (FIG. 20), respectively, generated from an analytical model that is based on previously obtained vasculature and hemodynamic phenomenological empirical measurements, of a plurality of hemodialysis patients previously treated for having AVFs exhibiting high venous blood flow or/and pressure.

Highlights and main findings of, and guidance provided by, the data and information presented in FIGS. 17-20 are as follows. Focus is on the previously determined guidance (based on review and analysis of the data and information generated and presented in FIGS. 13-16) for a reduced (reconstructed) AVF venous segment lumen diameter to be in the range of between about 8 mm (0.8 cm) and about 4 mm (0.4 cm), and also on the generally accepted guideline that within a typical hemodialysis access (such as an arteriovenous fistula (AVF) access), normal venous blood volumetric flow rate is in the range of between about 0.5 liter/min-1.5 liter/min (500 mL/min-1500 mL/min):
1) For an initial AVF venous segment blood volumetric flow rate of 2 liter/min (FIGS. 17 and 19), for establishing a normal AVF venous blood volumetric flow rate in the range of between about 0.5 liter/min-1.5 liter/min, the desired AVF reconstructed venous segment lumen diameter is in the range of between about 7.5 mm and about 5.5 mm, as indicated in FIGS. 17 and 19 by the dashed lines portion 356 and arrows 358 and 360.
   This finding leads to the guidance that for implementing the AVF dilated venous segment treating method (for example, method 100, as illustratively described herein with reference to FIGS. 1A-11b), particularly, step (procedure) 102 [102d, 102e], the AVF dilated venous segment lumen diameter should be reduced (reconstructed, re-sectioned) such that the resulting AVF reconstructed venous segment lumen diameter will be in the range of between about 7.5 mm (0.75 cm) and about 5.5 mm (0.55 cm), without causing undesirable significant increase in AVF mean arterial blood pressure [entering the AVF reconstructed venous segment] in the hemodialysis patient.
2) For an initial AVF venous segment blood volumetric flow rate of 4 liter/min (FIGS. 18 and 20), for establishing a normal AVF venous blood volumetric flow rate in the range of between about 0.5 liter/min-1.5 liter/min, the desired AVF reconstructed venous segment lumen diameter is in the range of between about 6.5 mm and about 4.5 mm, as indicated in FIGS. 18 and 20 by the dashed lines portion 362 and arrows 364 and 366.
   This finding leads to the guidance that for implementing the AVF dilated venous segment treating method (for example, method 100, as illustratively described herein with reference to FIGS. 1A-11b), particularly, step (procedure) 102 [102d, 102e], the AVF dilated venous segment lumen diameter should be reduced (reconstructed, re-sectioned) such that the resulting AVF reconstructed venous segment lumen diameter will be in the range of between about 6.5 mm (0.65 cm) and about 4.5 mm (0.45 cm), without causing undesirable significant increase in AVF mean arterial blood pressure [entering the AVF reconstructed venous segment] in the hemodialysis patient.
3) The results of (1) and (2) show that for establishing a normal AVF venous blood volumetric flow rate in the range of between about 0.5 liter/min-1.5 liter/min, the desired AVF reconstructed venous segment lumen diameter is slightly dependent on the initial AVF venous segment blood volumetric flow rate, at least, in the range of between 2 liter/min and 4 liter/min.

The above illustratively described highlights and main findings of, and guidance provided by, the data and information presented in FIGS. 13-20 are applicable for implementing the AVF reconstructed venous segment lumen diameter pre-determining method (for example, method 300, FIG. 12), as follows.

For example, according to step (procedure) 302, there is forming a reference set of previously obtained empirical measurements of venous blood volumetric flow rate and venous lumen diameter inside the AVF dilated venous segment of the hemodialysis patient. Such a reference set of previously obtained empirical measurements includes measurement of the initial venous segment blood volumetric flow rate inside the AVF dilated venous segment of the hemodialysis patient. For example, the initial venous segment blood volumetric flow rate inside the patient's AVF dilated venous segment may be equal to, or about (close to), 2 liter/min, or, alternatively, may be equal to, or about (close to), 4 liter/min.

Then, according to step (procedure) 304, there is using data and information generated from a quantitative mathematical analytical model, for analyzing the reference set of previously obtained empirical measurements of the hemodialysis patient, so as to identify the pre-determined AVF reconstructed venous segment lumen diameter that is suitable for treating the AVF dilated venous segment in the hemodialysis patient. Accordingly, for example, there is using data and information generated from the hereinabove described quantitative mathematical analytical model (for example, as presented in FIGS. 13-20), so as to identify the pre-determined AVF reconstructed venous segment lumen diameter that is suitable for treating the AVF dilated venous segment in the hemodialysis patient.

Specifically, for example, in a first exemplary scenario, wherein the initial venous segment blood volumetric flow rate inside the patient's AVF dilated venous segment being equal to, or about (close to), 2 liter/min, using and analyzing the data and information generated from the hereinabove described quantitative mathematical analytical model (for example, as presented in FIGS. 17 and 19), leads to identifying the pre-determined AVF reconstructed venous segment lumen diameter as being in the range of between about 7.5 mm (0.75 cm) and about 5.5 mm (0.55 cm), that is suitable for treating the AVF dilated venous segment in the hemodialysis patient.

Specifically, for example, in a second exemplary scenario, wherein the initial venous segment blood volumetric flow rate inside the patient's AVF dilated venous segment being equal to, or about (close to), 4 liter/min, using and analyzing the data and information generated from the hereinabove described quantitative mathematical analytical model (for example, as presented in FIGS. 18 and 20), leads to identifying the pre-determined AVF reconstructed venous segment lumen diameter as being in the range of between about 6.5 mm (0.65 cm) and about 4.5 mm (0.45 cm), that is suitable for treating the AVF dilated venous segment in the hemodialysis patient.

Then, according to step (procedure) 306, there is forming a reference set of previously obtained empirical measurements of mean arterial blood pressure that enters the AVF dilated venous segment of the hemodialysis patient, and analyzing the reference set of the mean arterial blood pressure, for checking and confirming the identified pre-determined AVF reconstructed venous segment lumen diameter as being suitable for treating the AVF dilated venous segment in the hemodialysis patient. Step (procedure) 306 is performed in order to ensure that, by subjecting the hemodialysis patient to the AVF dilated venous segment treating method, the resulting AVF reconstructed venous segment lumen diameter will not cause undesirable significant increase in AVF mean arterial blood pressure [entering the AVF reconstructed venous segment] in the hemodialysis patient.

In exemplary embodiments, the AVF reconstructed venous segment lumen diameter pre-determining method 300 is particularly applicable to the method (for example, method 100 presented in FIGS. 1A-11b) for treating an arteriovenous fistula dilated venous segment in a hemodialysis patient whose arteriovenous fistula exhibits abnormally high venous blood flow or/and pressure. For example, as shown in FIG. 12, in step (procedure) 320, there is using the identified pre-determined AVF reconstructed venous segment lumen diameter [from 304, 306] for: (i) forming the AVF reconstructed venous segment (via step 102 [102e] of method 100, including optional use of a venous segment lumen diameter forming device (LDFD)); and (ii) selecting the diameter of the blood vessel external support that is to be used for covering and supporting the AVF reconstructed venous segment (via step 104 [104a] of method 100) in the hemodialysis patient to be treated.

Specifically, for example, performing step (procedure) 306 may result in favorably checking and confirming that the identified pre-determined AVF reconstructed venous segment lumen diameter is suitable for treating the AVF dilated venous segment in the hemodialysis patient. Then, for example, per step (procedure) 320, there is using the (checked and confirmed) identified pre-determined AVF reconstructed venous segment lumen diameter (for example, in the range of between about 7.5 mm (0.75 cm) and about 5.5 mm (0.55 cm), per the above first exemplary scenario, or, alternatively, in the range of between about 6.5 mm (0.65 cm) and about 4.5 mm (0.45 cm), per the above second exemplary scenario), for: (i) forming the AVF reconstructed venous segment (via step 102 [102e] of method 100, including optional use of a venous segment lumen diameter forming device (LDFD)); and (ii) selecting the diameter of the blood vessel external support that is to be used for covering and supporting the AVF reconstructed venous segment (via step 104 [104a] of method 100) in the hemodialysis patient to be treated.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic/grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

The term ‘method’, as used herein, refers to a single step, procedure, manner, means, or/and technique, or a sequence, set, or group of two or more steps, procedures, manners, means, or/and techniques, for accomplishing or achieving a given task or action. Any such herein disclosed method, in a non-limiting manner, may include one or more steps, procedures, manners, means, or/and techniques, that are known or readily developed from one or more steps, procedures, manners, means, or/and techniques, previously taught about by practitioners in the relevant field(s) and art(s) of the herein disclosed invention. In any such herein disclosed method, in a non-limiting manner, the stated or presented sequential order of one or more steps, procedures, manners, means, or/and techniques, is not limited to that specifically stated or presented sequential order, for accomplishing or achieving a given task or action, unless otherwise specifically defined or stated herein, or, unless the context clearly dictates otherwise. Accordingly, in any such herein disclosed method, in a non-limiting manner, there may exist one or more alternative sequential orders of the same steps, procedures, manners, means, or/and techniques, for accomplishing or achieving a same given task or action, while maintaining same or similar meaning and scope of the herein disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, is considered equivalent to, and meaning the same as, the phrase ‘room temperature refers to a temperature in a range of from about 20° C. to about 25° C.’.

The term ‘about’, as used herein, refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, are encompassed by the broad scope of the appended claims.

All publications, patents, and or/and patent applications, cited or referred to in this disclosure are herein incorporated in their entirety by reference into the specification, to the same extent as if each individual publication, patent, or/and patent application, was specifically and individually indicated to be incorporated herein by reference. In addition, citation or identification of any reference in this specification shall not be construed or understood as an admission that such reference represents or corresponds to prior art of the present invention. To the extent that section headings are used, they should not be construed as necessarily limiting.

What is claimed is:

1. A method for treating an arteriovenous fistula dilated venous segment in a hemodialysis patient whose arteriovenous fistula exhibits abnormally high venous blood flow or/and pressure, the method comprising:
surgically reconstructing the arteriovenous fistula dilated venous segment, for forming an arteriovenous fistula reconstructed venous segment; and
covering and supporting said arteriovenous fistula reconstructed venous segment with an implantable blood vessel external support, such that the arteriovenous fistula exhibits normal venous blood flow and pressure.

2. The method of claim 1, wherein said surgically reconstructing the arteriovenous fistula dilated venous segment includes radially cutting through an end portion of the arteriovenous fistula dilated venous segment, so as to open and disconnect said arteriovenous fistula dilated venous segment end from the adjacent remaining venous portion of the arteriovenous fistula, thereby forming an opened and disconnected end of the arteriovenous fistula dilated venous segment.

3. The method of claim 2, wherein said radially cutting is performed at either a proximal location or at a distal location relative to the midline of the patient's body, so as to form a corresponding proximal radial cut, or a distal radial cut, through the proximal end portion, or through the distal end portion, respectively, of the arteriovenous fistula dilated venous segment.

4. The method of claim 1, wherein said surgically reconstructing the arteriovenous fistula dilated venous segment includes longitudinally cutting along the arteriovenous fistula dilated venous segment, so as to form a longitudinal cut extending along the arteriovenous fistula dilated venous segment.

5. The method of claim 1, wherein said surgically reconstructing the arteriovenous fistula dilated venous segment includes performing a partial tissue reconstruction procedure on the arteriovenous fistula dilated venous segment, by removing venous material therefrom, so as to form said arteriovenous fistula reconstructed venous segment.

6. The method of claim 5, further comprising closing said arteriovenous fistula reconstructed venous segment, to a desired arteriovenous fistula reconstructed venous segment lumen diameter.

7. The method of claim 6, wherein said closing of said arteriovenous fistula reconstructed venous segment includes using a venous segment lumen diameter forming device, around which tissue of said arteriovenous fistula reconstructed venous segment is worked on, in order to facilitate forming of said desired arteriovenous fistula reconstructed venous segment lumen diameter.

8. The method of claim 7, wherein said venous segment lumen diameter forming device is a mandrel or mandrel-type device.

9. The method of claim 6, wherein said closing of said arteriovenous fistula reconstructed venous segment includes using results obtained by performing a procedure for pre-determining said desired arteriovenous fistula reconstructed venous segment lumen diameter.

10. The method of claim 1, wherein said covering and supporting said arteriovenous fistula reconstructed venous segment with said implantable blood vessel external support includes using results obtained by performing a procedure for pre-determining a desired arteriovenous fistula reconstructed venous segment lumen diameter for said arteriovenous fistula reconstructed venous segment.

11. The method of claim 1, wherein said implantable blood vessel external support comprises a braided tubular body having an inner diameter in a range of between 4 mm and 9 mm.

12. The method of claim 1, wherein said covering and supporting said arteriovenous fistula reconstructed venous segment with said implantable blood vessel external support includes performing anastomosis on said arteriovenous fistula reconstructed venous segment, by anastomotically connecting an opened and disconnected end of said arteriovenous fistula reconstructed venous segment to the adjacent remaining venous portion of the arteriovenous fistula, so as to form an anastomosis site that connects said arteriovenous fistula reconstructed venous segment to the arteriovenous fistula, such that said arteriovenous fistula reconstructed venous segment becomes a longitudinally continuous, integral part of the arteriovenous fistula.

13. The method of claim 12, further comprising making final adjustments of configuration of said blood vessel external support fitted over said arteriovenous fistula reconstructed venous segment, such that said blood vessel external support fully covers and supports entire lengths of said arteriovenous fistula reconstructed venous segment and of said anastomosis site.

14. A method for pre-determining an arteriovenous fistula reconstructed venous segment lumen diameter, whereby the pre-determined arteriovenous fistula reconstructed venous segment lumen diameter is to be used as guidance for treating an arteriovenous fistula dilated venous segment in a hemodialysis patient whose arteriovenous fistula exhibits abnormally high venous blood flow or/and pressure, the method comprising:
forming a reference set of previously obtained empirical measurements of venous blood volumetric flow rate and venous lumen diameter inside the arteriovenous fistula dilated venous segment of the hemodialysis patient; and
using data and information generated from a mathematical quantitative analytical model, for analyzing said reference set of previously obtained empirical measurements of the hemodialysis patient, so as to identify the pre-determined arteriovenous fistula reconstructed venous segment lumen diameter that is suitable for treating the arteriovenous fistula dilated venous segment in the hemodialysis patient.

15. The method of claim 14, further comprising:
forming a reference set of previously obtained empirical measurements of mean arterial blood pressure that enters the arteriovenous fistula dilated venous segment of the hemodialysis patient; and
using said reference set of said mean arterial blood pressure, for checking and confirming said identified pre-determined arteriovenous reconstructed venous segment lumen diameter as being suitable for treating the arteriovenous fistula dilated venous segment in the hemodialysis patient.

16. The method of claim 14, wherein said using data and information generated from said mathematical quantitative analytical model includes mathematically quantitatively analytically modelling said previously obtained vasculature and hemodynamic phenomenological empirical measurements made on hemodialysis patients treated for having arteriovenous fistulas exhibiting high venous blood flow or/and pressure.

17. The method of claim 14, wherein said mathematical quantitative analytical model is used for generating said data and information in a form of values of arteriovenous fistula mean arterial blood pressure as a function of arteriovenous fistula venous segment lumen diameter, and in a form of values of arteriovenous fistula venous segment blood volumetric flow rate as a function of arteriovenous fistula venous segment lumen diameter.

18. The method of claim 14, wherein said identified pre-determined arteriovenous fistula reconstructed venous segment lumen diameter is used as guidance in a procedure for surgically reconstructing the arteriovenous fistula dilated venous segment, for forming an arteriovenous fistula reconstructed venous segment in the hemodialysis patient.

19. The method of claim 18, wherein said identified pre-determined arteriovenous fistula reconstructed venous segment lumen diameter is used as guidance in a procedure for selecting an inner diameter of a venous segment lumen diameter forming device, around which tissue of said arteriovenous fistula reconstructed venous segment is worked on, in order to facilitate forming of a desired arteriovenous fistula reconstructed venous segment lumen diameter.

20. The method of claim 18, wherein said identified pre-determined arteriovenous fistula reconstructed venous segment lumen diameter is further used as guidance in a procedure for covering and supporting said arteriovenous fistula reconstructed venous segment with an implantable blood vessel external support, such that the arteriovenous fistula exhibits normal venous blood flow and pressure.

21. The method of claim 18, wherein said identified pre-determined arteriovenous fistula reconstructed venous segment lumen diameter is used as guidance in a procedure for selecting a diameter of a blood vessel external support for covering and supporting said arteriovenous fistula reconstructed venous segment in the hemodialysis patient.

22. A method of using an implantable blood vessel external support in the treatment of a hemodialysis arteriovenous fistula dilated venous segment, in a hemodialysis patient whose arteriovenous fistula exhibits abnormally high venous blood flow or/and pressure, the method comprising:
surgically reconstructing the arteriovenous fistula dilated venous segment, for forming an arteriovenous fistula reconstructed venous segment; and
covering and supporting said arteriovenous fistula reconstructed venous segment with the implantable blood vessel external support, such that the arteriovenous fistula exhibits normal venous blood flow and pressure.
